(19) United States
(12) Patent Application Publication
(10) Pub. No.: US 2020/0023005 A1
(43) Pub. Date: Jan. 23, 2020

(54) MITOCHONDRIAL COMPOSITIONS AND METHODS FOR TREATMENT OF SKIN AND HAIR

(71) Applicant: MINOVIA THERAPEUTICS LTD., Tirat HaCarmel (IL)

(72) Inventors: Natalie YIVGI-OHANA, Haifa (IL); Uriel HALAVEE, Tel Aviv (IL); Shmuel BUKSHPAN, Ramat Hasharon (IL)

(21) Appl. No.: 16/497,573

(22) PCT Filed: Mar. 22, 2018
(86) PCT No.: PCT/IL2018/050332

§ 371 Date: Sep. 25, 2019

Related U.S. Application Data
(60) Provisional application No. 62/476,792, filed on Mar. 26, 2017.

(51) Int. Cl. A61K 35/14; A61P 17/14; A61K 9/06; A61K 35/50; A61K 9/00; A61Q 17/04; A61K 8/9789; A61K 8/98; A61K 8/60

(52) U.S. Cl. CPC A61K 35/14; A61P 17/14; A61K 9/06; A61K 35/50; A61K 8/60; A61Q 17/04; A61K 8/9789; A61K 8/983; A61K 8/982; A61K 9/0014

(57) ABSTRACT

The present invention relates to products and methods for treatment of hair loss. Specifically, the present application relates to compositions and methods for prevention and treatment of hair loss, comprising administration of a composition comprising intact mitochondria, ruptured mitochondria and/or mitochondrial constituents.

### FIELD OF THE INVENTION

The present invention relates to compositions and methods for treatment of hair-producing organs and cells, such as hair follicles. Specifically, the present application relates to compositions and formulations comprising intact and/or ruptured mitochondria, and to methods for the prevention and treatment of hair loss.

### BACKGROUND OF THE INVENTION

A hair follicle, situated within the skin, is the hair-producing organ. The hair follicle comprises several parts, including the papilla, the hair matrix around the papilla, the root sheath and the bulge. Dermal papilla cells are found at the bottom of the follicle, while the follicular matrix surrounds the papilla, the root sheath and the hair fiber.

Hair grows in cycles of several phases termed anagen (the growth phase), catagen (the involuting or regressing phase) and telogen (the resting or quiescent phase). Normally up to 85-90% of the hair follicles are in anagen phase, 10-14% are in telogen phase and 1-2% are in catagen phase.

Anagen is the active growth phase of hair follicles. During this phase the hair grows at a rate of about 1 centimeter every 28 days. Scalp hair stays in this active phase of growth for 2-7 years. The amount of time the hair follicle stays in the anagen phase is genetically determined. At the end of the anagen phase an unknown signal causes the follicle to go into the catagen phase. The catagen phase lasts for about 2-3 weeks while the hair is cut off from its blood supply. At the end of the catagen phase, the hair follicle enters the resting telogen phase. At the end of the telogen phase the hair follicle re-enters the anagen phase. The dermal papilla and the base of the follicle re-join and a new hair is formed. If the prior hair has not already been shed, the new hair pushes the old one out and the growth cycle re-starts.

Hair loss affects millions of people, including over 40% of men over the age of 30. Zheng and coworkers have identified an mtDNA 4977 bp deletion in hair samples, and that the deletion loads increased from 0 to 1.436% of the total mtDNA with an exponential increase with age (Bosn. J. Basic Med. Sci., 2012, Vol. 12 (3), pages 187-192). Numerous other factors can cause hair loss, including genetic predisposition, autoimmune reactions, scarring, diseases and infection. Hair loss can ultimately lead to complete baldness. Alopecia is a medical condition in which hair is lost from an area of the body. Alopecia includes androgenetic alopecia, also known as male pattern baldness, and alopecia areata, which is thought to be an autoimmune disorder.

One symptom of alopecia is hair follicle miniaturization. In the miniaturization process, the hair follicle enters a prolonged lag phase following the telogen stage. With successive anagen cycles, the follicles become smaller and smaller, leading to shorter, finer hair. The miniaturized follicle eventually produces a tiny hair shaft that is visually insignificant. Ultimately, the follicle may stop producing hair shafts and the area of hair loss can become completely devoid of hair.

Several methods for treating hair loss are available, including drugs such as topical Minoxidil and orally-delivered Finasteride. However, these treatments have achieved limited success in restoring natural hair growth and may cause various unwanted side effects. A surgical treatment for hair loss is hair follicle transplantation, a procedure in which hair follicles are transplanted from a non-balding region of the scalp to a region of hair loss.

The exact mechanism(s) in which Minoxidil and Finasteride modulate hair loss are not fully elucidated, but it is thought that Minoxidil mainly shortens telogen while Finasteride mainly increases anagen (Messenger A. G. et al., Minoxidil: Mechanisms of action on hair growth, British Journal of Dermatology, 2004, (150):186-194; Van Neste D. et al., Finasteride increases anagen hair in men with androgenetic alopecia, British Journal of Dermatology, 2000, (143):804-810).

U.S. Pat. No. 7,279,326 relates to a composition for delivering a wild-type mitochondrial DNA genome to a mammalian cell. U.S. Pat. No. 9,603,872 relates to methods, kits and compositions for mitochondrial replacement in the treatment of disorders arising from mitochondrial dysfunction. U.S. 2004/0122109 relates to preparation for growing hair and preventing hair removal. DE 10 2013 225 588 relates to cosmetic, non-therapeutic use of hair treatment composition comprising extract of chicory.

Despite progress in the field, there still remains an unmet need for a safe and efficient therapy for preventing, treating and reversing hair loss.

### SUMMARY OF THE INVENTION

The present invention provides compositions, formulations and methods for prevention or treatment of hair loss. The present invention is based in part on the unexpected positive effect of mitochondrial constituents on hair follicle elongation and on function of dermal papilla cells.

Without wishing to be bound by any theory or mechanism, treating hair follicles according to the methods of the present invention may facilitate hair growth in subjects in need thereof, such as, but not limited to, subjects suffering from alopecia. Using intact mitochondria and/or ruptured mitochondria and/or mitochondrial constituents for treating a subject afflicted with alopecia, according to the method of the present invention, may be safer than using other known methods, as mitochondria or mitochondrial constituents are of a physiological origin.

The present invention provides, in one aspect, a cosmetic composition for preventing, ameliorating or treating hair loss, wherein the composition comprises an effective amount of about 1 μg/ml to about 100 μg/ml of intact mitochondria, ruptured mitochondria and/or mitochondrial constituents selected from the group consisting of mitochondrial protein, mitochondrial nucleic acid, mitochondrial lipid and mitochondrial saccharide, wherein one or more of the intact mitochondria, the ruptured mitochondria and/or the mitochondrial constituents is frozen or thawed, and wherein the composition further comprises a cosmetically-acceptable carrier and is formulated for topical administration to human skin.

In certain embodiments, the composition comprises about 5 μg/ml to about 90 μg/ml of the mitochondrial constituents, about 5 μg/ml to about 80 μg/ml of the mitochondrial constituents, about 5 μg/ml to about 70 μg/ml of the mitochondrial constituents, about 5 μg/ml to about 60 μg/ml of the mitochondrial constituents, or about 5 μg/ml to about 50 μg/ml of the mitochondrial constituents. In certain embodiments, the composition comprises about 12.5 μg/ml of the mitochondrial constituents. Each possibility represents a separate embodiment of the invention.

In certain embodiments, the composition comprises at least about 1 μg/ml of the mitochondrial constituents, at least about 3 μg/ml of the mitochondrial constituents, at least about 5 μg/ml of the mitochondrial constituents, at least about 10 μg/ml of the mitochondrial constituents, at least about 20 μg/ml of the mitochondrial constituents, at least about 30 μg/ml of the mitochondrial constituents, at least about 40 μg/ml of the mitochondrial constituents, or at least about 50 μg/ml of the mitochondrial constituents. Each possibility represents a separate embodiment of the invention.

In certain embodiments, the composition is frozen. In certain embodiments, the composition is frozen at 0° C. or at a lower temperature. In certain embodiments, the composition is frozen at −20° C. or at a lower temperature. In certain embodiments, the composition is frozen at −70° C. or at a lower temperature. In certain embodiments, the composition is frozen in liquid nitrogen.

In certain embodiments, the composition is thawed after being frozen. In certain embodiments, the composition is thawed and is at room temperature. In certain embodiments, the composition is thawed and is at a temperature of 15° C. to 30° C. In certain embodiments, the composition is thawed and is at 4° C.

In certain embodiments, at least part of the mitochondrial constituents is comprised in intact mitochondria. In certain embodiments, at least 5% of the mitochondrial constituents are comprised in intact mitochondria. In certain embodiments, at least 10% of the mitochondrial constituents are comprised in intact mitochondria. In certain embodiments, at least 20% of the mitochondrial constituents are comprised in intact mitochondria. In certain embodiments, at least 40% of the mitochondrial constituents are comprised in intact mitochondria. In certain embodiments, at least 60% of the mitochondrial constituents are comprised in intact mitochondria.

In certain embodiments, at least part of the mitochondrial constituents is comprised in ruptured mitochondria. In certain embodiments, at least 5% of the mitochondrial constituents are comprised in ruptured mitochondria. In certain embodiments, at least 10% of the mitochondrial constituents are comprised in ruptured mitochondria. In certain embodiments, at least 20% of the mitochondrial constituents are comprised in ruptured mitochondria. In certain embodiments, at least 40% of the mitochondrial constituents are comprised in ruptured mitochondria. In certain embodiments, at least 60% of the mitochondrial constituents are comprised in ruptured mitochondria.

In certain embodiments, the composition comprises a sugar. In certain embodiments, the composition comprises sucrose. In certain embodiments, the composition comprises about 10 mM to about 1000 mM sucrose. In certain embodiments, the composition comprises about 100 mM to about 400 mM sucrose. In certain embodiments, the composition comprises about 200 mM to about 250 mM sucrose. In certain embodiments, the composition comprises at least about 10 mM sucrose. In certain embodiments, the composition comprises at least about 100 mM sucrose. In certain embodiments, the composition comprises at least about 200 mM sucrose.

In certain embodiments, the composition comprises sucrose, EGTA/Tris and Tris/MOPS. In certain embodiments, the composition comprises about 200 mM to about 250 mM sucrose, about 1 mM EGTA/Tris and about 10 mM Tris/MOPS.

In certain embodiments, the intact mitochondria, ruptured mitochondria and/or mitochondrial constituents are obtainable or obtained by a method performed ex-vivo. In certain embodiments, the intact mitochondria, ruptured mitochondria and/or mitochondrial constituents are obtainable or obtained by a method comprising the steps (a)-(d): (a) obtaining a sample of a human or plant tissue or organ, (b) homogenizing the tissue or organ, (c) isolating the liquid phase, and (d) isolating intact mitochondria, ruptured mitochondria and/or mitochondrial constituents from the liquid phase. In certain embodiments, the method further comprises step (e) freezing the intact mitochondria, ruptured mitochondria and/or mitochondrial constituents. In certain embodiments, the liquid phase in step (c) of the method is isolated by centrifugation at 600 g for 10 minutes at 4° C. and/or by filtration through a 5 μm cutoff filter. In certain embodiments, the intact mitochondria, ruptured mitochondria and/or mitochondrial constituents in step (d) of the method are isolated from the liquid phase by centrifugation at 7000 g for 15 minutes at 4° C. Each possibility represents a separate embodiment of the invention.

In certain embodiments, the composition is not formulated in water. In certain embodiments, the composition is not formulated as an aqueous solution. In certain embodiments, the composition is not formulated as a suspension. In certain embodiments, the composition is formulated as a colloid, lotion, cream, ointment, foam or gel. Each possibility represents a separate embodiment of the invention.

The present invention further provides, in another aspect, a personal hygiene, a skin or a hair product, comprising any one of the compositions or formulations described herein.

In certain embodiments, the product is frozen. In certain embodiments, the product is thawed. In certain embodiments, the product is a lotion, a cream, an ointment, foam, a gel, a soap, a shampoo or a conditioner.

The present invention further provides, in yet another aspect, a method for preventing, ameliorating or treating hair loss, the method comprising administering to a subject in need thereof a composition comprising an effective amount of about 1 μg/ml to about 100 μg/ml of intact mitochondria, ruptured mitochondria and/or mitochondrial constituents selected from the group consisting of mitochondrial protein, mitochondrial nucleic acid, mitochondrial lipid and mitochondrial saccharide.

In certain embodiments, the treatment is selected from the group consisting of stopping hair follicle miniaturization, slowing hair follicle miniaturization, reversing hair follicle miniaturization, inducing elongation of hair follicles, enhancing elongation of hair follicles, inducing proliferation of cells within hair follicles, enhancing proliferation of cells within hair follicles, inducing elongation of hair fibers, enhancing elongation of hair fibers, enhancing thickness of hair fibers, altering the duration of hair follicle growth cycle phases and any combination thereof. Each possibility represents a separate embodiment of the invention.

In certain embodiments, the treatment is inducing or enhancing elongation of hair follicles. In certain embodiments, the treatment is inducing or enhancing proliferation of cells within hair follicles.

In certain embodiments, the intact mitochondria, ruptured mitochondria and/or mitochondrial constituents are thawed.

In certain embodiments, the composition is formulated as a colloid, a liquid, a lotion, a cream, an ointment, foam or gel.

In certain embodiments, the mitochondria are derived from a cell or a tissue selected from the group consisting of placenta, placental cells grown in culture, blood cells, plant tissue, plant cells and plant cells grown in culture. Each possibility represents a separate embodiment of the invention. In certain embodiments, the plant tissue is potato tissue or sprout tissue. In certain embodiments, the plant cells are potato cells or sprout cells.

In certain embodiments, the composition is administered by topical administration, oral administration, subcutaneous administration, intradermal administration, transdermal administration or systemic administration. Each possibility represents a separate embodiment of the present invention. In certain embodiments, the composition is administered by topical administration. In certain embodiments, the composition is administered by systemic administration. In certain embodiments, the composition is administered to a human scalp.

In certain embodiments, the subject is afflicted with a disease, disorder or condition which has a deleterious effect on hair vitality. In certain embodiments, the subject is afflicted with alopecia. In certain embodiments, the subject is afflicted with a mitochondrial disease. In certain embodiments, the mitochondrial disease is a deletion in mitochondrial DNA. In certain embodiments, the deletion in mitochondrial DNA is a 4977 bp deletion. In certain embodiments, the mitochondrial disease is Pearson syndrome. In certain embodiments, the subject is afflicted with cancer. In certain embodiments, the subject is treated by radiation or is to be treated by radiation. In certain embodiments, the subject is treated by chemotherapy or is to be treated by chemotherapy. In certain embodiments, the subject is afflicted with an autoimmune disorder. In certain embodiments, the autoimmune disorder is alopecia areata.

In certain embodiments, the subject is over 30, over 40, over 50 or over 60 years of age. Each possibility represents a separate embodiment of the present invention. In certain embodiments, the subject is a male.

According to another aspect, the present invention provides a method for treatment of hair follicles, the method comprising administering to a subject in need thereof an effective amount of a composition comprising intact mitochondria and/or ruptured mitochondria.

According to another aspect, the present invention provides a method for treatment of alopecia, the method comprising administering to a subject afflicted with alopecia an effective amount of a composition comprising intact mitochondria and/or ruptured mitochondria.

According to some embodiments, the mitochondrial constituent is selected from the group consisting of: mitochondrial protein, mitochondrial nucleic acid, mitochondrial lipid, mitochondrial saccharide, mitochondrial structure, at least part of a mitochondrial matrix and a combination thereof. Each possibility represents a separate embodiment of the present invention.

According to some embodiments, the composition comprising the intact mitochondria further comprises a hypertonic solution. According to some embodiments, the hypertonic solution comprises a saccharide. According to another embodiment, the hypertonic solution comprises sucrose.

According to another embodiment, the concentration of the composition is up to about 50 μg/ml. According to another embodiment, the concentration of the composition is between about 10-20 μg/ml. According to another embodiment, the concentration of the composition is between about 10-15 μg/ml. According to another embodiment, the concentration of the composition is about 12.5 μg/ml. According to another embodiment, the concentration of the composition is between about 5-50 μg/ml.

According to another embodiment, the method of the invention further comprises administration of at least one hair-growth inducing agent. According to some embodiments, the at least one hair-growth inducing agent is selected from the group consisting of: Finasteride, Dutasteride, Minoxidil, Kopexil, oxidized coenzyme Q, reduced coenzyme Q, L-Carnitine-Tartrate, caffeine and a combination thereof. Each possibility represents a separate embodiment of the present invention.

According to another embodiment, the cell or tissue are from a source selected from allogeneic and xenogeneic.

According to another embodiment, the subject is afflicted with a disease or disorder which would benefit from treatment of hair follicles. According to another embodiment, the disease or disorder which would benefit from treatment of hair follicles is alopecia.

Further embodiments, features, advantages and the full scope of applicability of the present invention will become apparent from the detailed description and drawings given hereinafter. However, it should be understood that the detailed description, while indicating embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a bar graph comparing the elongation of untreated (Vehicle) human hair follicles (hHFs) to the elongation of hHFs treated with Cyclosporine A, or with various concentrations of frozen-thawed human placental mitochondrial compositions according to the present invention.

FIGS. 2A-2C are bar graphs comparing the effect of a fresh human placental mitochondrial composition according to the present invention on human dermal papilla cell number (FIG. 2A), VEGF secretion (FIG. 2B) and citrate synthase activity (FIG. 2C).

FIG. 3 is a dot-plot graph showing O₂ consumption (via fluorescence) over time in fresh and frozen-thawed murine placental mitochondria.

FIGS. 4A-4D are dot-plot graphs comparing oxygen consumption (via fluorescence) of fresh potato mitochondria incubated in a buffer containing mannitol (FIG. 4A) or sucrose (FIG. 4B), to the oxygen consumption of the corresponding mitochondria following a freeze-thaw cycle (FIG. 4C and FIG. 4D, respectively).

FIG. 5 is a bar graph comparing the release of citrate synthase from fresh and thawed potato mitochondria incubated in a buffer comprising mannitol or sucrose.

FIGS. 6A-6B are dot-plot graphs comparing oxygen consumption (via fluorescence) of murine placenta mitochondria incubated in isolation buffer (FIG. 6A) or PBS (FIG. 6B).

FIG. 6C is a bar graph comparing citrate synthase release from mitochondria incubated in isolation buffer or PBS.

FIG. 7A is a dot plot graph comparing oxygen consumption (via fluorescence) of murine placenta mitochondria incubated in isolation buffer or OptiMEM cell medium (Gibco). FIG. 7B is a bar graph comparing citrate synthase release from mitochondria incubated in isolation buffer or OptiMEM (Gibco).

FIG. 8 is a bar graph comparing progesterone secretion to the medium of human term placental mitochondria at start (T0) and after incubation for 24 hours (T24 h).

FIG. 9 is a bar graph comparing ATP levels in human follicle dermal papilla cells (hFDPCs) before and after treatment with sprout mitochondria.

FIGS. 10A-10C are bar graphs presenting the effect of human placenta mitochondria in human follicle dermal papilla cells (hFDPCs) on citrate synthase (CS) enzyme activity (FIG. 10A), cell proliferation (FIG. 10B), and VEGF secretion (FIG. 10C).

FIGS. 11A-11D are bar graphs presenting the effect of sprout mitochondria in human skin (hFDPCs) on the UV-B-induced production of ROS (FIG. 11A and FIG. 11B) and IL-1α (FIG. 11C and FIG. 11D).

### DETAILED DESCRIPTION OF THE INVENTION

The present invention discloses for the first time compositions, formulations, products and methods for treating hair follicles thereby improving hair vitality. The present invention is based in part on the unexpected beneficial effect of mitochondria and/or mitochondrial constituents on elongation of human hair follicles and on proliferation of human follicular dermal papilla cells, as exemplified herein below. Without wishing to be bound by any theory or mechanism, using the products and methods of the invention results in a higher number of longer and thicker hair and in less hair shedding in the treated area.

The present invention provides, in one aspect, a composition comprising an effective amount of about 1 μg/ml to about 100 μg/ml of intact mitochondria, ruptured mitochondria and/or mitochondrial constituents selected from the group consisting of mitochondrial protein, mitochondrial nucleic acid, mitochondrial lipid and mitochondrial saccharide, wherein one or more of the intact mitochondria, the ruptured mitochondria and/or the mitochondrial constituents is frozen or thawed, and wherein the composition further comprises a carrier and is formulated for topical administration to human skin.

According to the principles of the present invention, the compositions provided herein are mainly intended for cosmetic purposes. In certain embodiments, the compositions and/or formulations and/or products of the invention are cosmetic and comprise a cosmetically-acceptable carrier. However, these compositions may also be employed in therapeutic methods. In certain embodiments, the compositions and/or formulations and/or products of the invention are pharmaceutical and/or therapeutic and comprise a pharmaceutically-acceptable carrier.

The term “cosmetic composition” as used herein relates to a composition suitable for application to the human body. The term “cosmetically acceptable carrier” refers to all carriers and/or excipients and/or diluents conventionally used in cosmetic compositions. The term “pharmaceutical composition” as used herein relates to a composition suitable for application to the human body. The term “pharmaceutically acceptable carrier” refers to all carriers and/or excipients and/or diluents conventionally used in pharmaceutical compositions. The term “topical administration” as used herein refers to administration to body surfaces.

The term “cosmetic” or “cosmetic composition” as used herein is further intended to include all types of products which are applied in any manner directly to the subject and is intended to include, in addition to the cosmetic agent invention disclosed herein, conventional ingredients such as lanolin, beeswax, oleic acid, spermaceti, almond oil, castor oil, tragacanth gum, clay, magnesia, talc, metal stearates, chalk, magnesium carbonate, zinc stearate, kaolin, etc. These compositions may take the form of fatty or non-fatty creams, milky suspensions or emulsions of the water-in-oil or oil-in-water types, lotions, gels or jellies, colloidal or non-colloidal aqueous or oily solutions, pastes, soaps, aerosols, soluble tablets (to be dissolved in a fluid, such as water) or sticks. The cosmetic compositions according to the invention may also contain conventional vehicles or carriers, such as solvents, fats, oils and mineral waxes, fatty acids and derivatives thereof, alcohols and derivatives thereof, glycols and derivatives thereof, glycerol and derivatives thereof, sorbitol and derivatives thereof, surface-active agents of the anionic, cationic or nonionic type, emulsifying agents, preserving agents, perfumes, etc.

As used herein, the term “the composition” and “the composition of the invention” are used interchangeably. According to some embodiments, the term “the composition of the invention”, as used herein, refers to a composition comprising intact mitochondria and/or ruptured mitochondria and/or mitochondrial constituents. According to some embodiments, the term “the composition of the invention”, as used herein, refers to mitochondria selected from the group consisting of: intact mitochondria and ruptured mitochondria. According to some embodiments, the composition of the invention comprises ruptured mitochondria. According to some embodiments, the composition of the invention comprises intact mitochondria. According to other embodiments, the composition of the invention comprises intact mitochondria and ruptured mitochondria. According to some embodiments, the composition of the invention comprises at least one mitochondrial constituent. According to some embodiments, the composition of the invention comprises ruptured mitochondria and at least one mitochondrial constituent. According to some embodiments, the composition of the invention comprises ruptured mitochondria and at least one mitochondrial constituent released and/or secreted from the ruptured mitochondria. Each possibility represents a separate embodiment of the present invention. According to some embodiments, the composition of the invention comprises partially purified mitochondria. According to some embodiments, the composition of the invention comprises isolated mitochondria. According to some embodiments, the composition of the invention comprises a medium conditioned by mitochondria. According to other embodiments, the composition of the invention comprises at least one of the group consisting of: ruptured mitochondria, at least one mitochondrial constituent, isolated mitochondria, partially purified mitochondria, intact mitochondria, a media conditioned by mitochondria and a combination thereof. Each possibility represents a separate embodiment of the present invention. As used herein, the term “medium conditioned by mitochondria” refers to a medium in which mitochondria were incubated and which contains mitochondrial constituents and/or elements secreted from mitochondria.

In certain embodiments, the intact mitochondria, ruptured mitochondria and/or mitochondrial constituents are frozen. In certain embodiments, the intact mitochondria, ruptured mitochondria and/or mitochondrial constituents are thawed. The term “thawed” as used herein means have undergone at least one freeze-thaw cycle, e.g. were at least once frozen but now un-frozen.

In certain embodiments, the composition comprises about 5 μg/ml to about 90 μg/ml of the mitochondrial constituents, about 5 μg/ml to about 80 μg/ml of the mitochondrial constituents, about 5 μg/ml to about 70 μg/ml of the mitochondrial constituents, about 5 μg/ml to about 60 μg/ml of the mitochondrial constituents, about 5 μg/ml to about 50 μg/ml of the mitochondrial constituents or about 5 μg/ml to about 30 μg/ml of the mitochondrial constituents. In certain embodiments, the composition comprises about 12.5 μg/ml of the mitochondrial constituents. As used herein, the term “about” refers to +/−10% of the indicated value.

According to some embodiments, the total concentration of all of the mitochondrial constituents in the composition of the invention is between about 10-20 μg/ml. According to other embodiments, the concentration is between about 10-15 μg/ml. According to some embodiments, the concentration is about 12.5 μg/ml. According to some embodiments, the concentration is at least about 10 μg/ml. According to some embodiments, the concentration is at least about 12.5 μg/ml. According to some embodiments, the concentration is no more than about 50 μg/ml. According to some embodiments, the concentration is between about 1-50 μg/ml. According to some embodiments, the concentration is between about 5-50 μg/ml. According to some embodiments, the concentration is between about 0.1-50 μg/ml. According to some embodiments, the concentration is no more than about 100 μg/ml.

In certain embodiments, the composition comprises at least about 1 μg/ml of the mitochondrial constituents, at least about 3 μg/ml of the mitochondrial constituents, at least about 5 μg/ml of the mitochondrial constituents, at least about 10 μg/ml of the mitochondrial constituents, at least about 20 μg/ml of the mitochondrial constituents, at least about 30 μg/ml of the mitochondrial constituents, at least about 40 μg/ml of the mitochondrial constituents, or at least about 50 μg/ml of the mitochondrial constituents.

In certain embodiments, the composition is frozen. In certain embodiments, the composition is frozen at 0° C. or at a lower temperature. In certain embodiments, the composition is frozen at −20° C. or at a lower temperature. In certain embodiments, the composition is frozen at −70° C. or at a lower temperature. In certain embodiments, the composition is frozen in liquid nitrogen.

In certain embodiments, the composition is thawed after being frozen. In certain embodiments, the composition is thawed and is at room temperature. In certain embodiments, the composition is thawed and is at a temperature of 15° C. to 30° C. In certain embodiments, the composition is thawed and is at 4° C.

In certain embodiments, at least part of the mitochondrial constituents is comprised in intact mitochondria. In certain embodiments, at least 5% of the mitochondrial constituents are comprised in intact mitochondria. In certain embodiments, at least 10% of the mitochondrial constituents are comprised in intact mitochondria. In certain embodiments, at least 20% of the mitochondrial constituents are comprised in intact mitochondria. In certain embodiments, at least 40% of the mitochondrial constituents are comprised in intact mitochondria. In certain embodiments, at least 50% of the mitochondrial constituents are comprised in intact mitochondria.

In certain embodiments, at least part of the mitochondrial constituents is comprised in ruptured mitochondria. In certain embodiments, at least 5% of the mitochondrial constituents are comprised in ruptured mitochondria. In certain embodiments, at least 10% of the mitochondrial constituents are comprised in ruptured mitochondria. In certain embodiments, at least 20% of the mitochondrial constituents are comprised in ruptured mitochondria. In certain embodiments, at least 40% of the mitochondrial constituents are comprised in ruptured mitochondria. In certain embodiments, at least 50% of the mitochondrial constituents are comprised in ruptured mitochondria.

In certain embodiments, the composition comprises a sugar. In certain embodiments, the composition comprises sucrose. In certain embodiments, the composition comprises about 10 mM to about 1000 mM sucrose. In certain embodiments, the composition comprises about 100 mM to about 400 mM sucrose. In certain embodiments, the composition comprises about 200 mM to about 250 mM sucrose. In certain embodiments, the composition comprises at least about 10 mM sucrose. In certain embodiments, the composition comprises at least about 100 mM sucrose. In certain embodiments, the composition comprises at least about 200 mM sucrose.

In certain embodiments, the composition comprises sucrose, EGTA/Tris and Tris/MOPS. In certain embodiments, the composition comprises about 200 mM to about 250 mM sucrose, about 1 mM EGTA/Tris and about 10 mM Tris/MOPS.

In certain embodiments, the intact mitochondria, ruptured mitochondria and/or mitochondrial constituents are obtainable or obtained by a method performed ex-vivo. In certain embodiments, the intact mitochondria, ruptured mitochondria and/or mitochondrial constituents are obtainable or obtained by a method comprising the steps (a)-(d): (a) obtaining a sample of a human or plant tissue or organ, (b) homogenizing the tissue or organ, (c) isolating the liquid phase, and (d) isolating intact mitochondria, ruptured mitochondria and/or mitochondrial constituents from the liquid phase. In certain embodiments, the method further comprises step (e) freezing the intact mitochondria, ruptured mitochondria and/or mitochondrial constituents. In certain embodiments, the liquid phase in step (c) of the method is isolated by centrifugation at 600 g for 10 minutes at 4° C. and/or by filtration through a 5 μm cutoff filter. In certain embodiments, the intact mitochondria, ruptured mitochondria and/or mitochondrial constituents in step (d) of the method are isolated from the liquid phase by centrifugation at 7000 g for 15 minutes at 4° C.

In certain embodiments, the composition is devoid of water. In certain embodiments, the composition is not formulated in water. In certain embodiments, the composition is not formulated as an aqueous solution. In certain embodiments, the composition is not formulated as a suspension. In certain embodiments, the composition is formulated as a colloid, lotion, cream, ointment, foam or gel.

The present invention further provides, in another aspect, a personal hygiene product, a skin product or a hair product, comprising any one of the compositions or formulations described herein.

In certain embodiments, the product is frozen. In certain embodiments, the product is thawed. In certain embodiments, the product is a lotion, a cream, an ointment, foam, a gel, a soap, a shampoo or a conditioner.

The term “lotion” as used herein refers to a low-viscosity topical preparation intended for application to skin. The term “cream” as used herein refers to all cosmetic materials which include, for instance, hand creams, cleansing creams, milky lotions, cold creams, hair creams, foundation creams, beauty washes, facial packs and the like. The term “ointment” embraces formulations (including creams) having oleaginous, water-soluble and emulsion-type bases, e.g., petrolatum, lanolin, polyethylene glycols, as well as mixtures thereof. The term “foam” as used herein refers to a substance formed by trapping pockets of gas in a liquid or solid. The term “gel” generally means a dispersion which has a high viscosity and no fluidity. The term “soap” is used herein in its popular sense, i. e., the alkali metal or alkanol ammonium salts of aliphatic, alkanes, or alkene monocarboxylic acids. The term “shampoo” refers to hair cleansing preparations which are to be applied to the hair and then rinsed away. The term “hair conditioner” as used herein refers to a hair care product that changes the texture and appearance of hair. Hair conditioners are often a viscous liquid that is applied and massaged into the hair. Hair conditioners are usually used after washing the hair with shampoo.

The present invention further provides, in yet another aspect, a method for preventing, ameliorating or treating hair loss in a subject in need thereof, the method comprising administering to the subject a composition comprising an effective amount of about 1 μg/ml to about 100 μg/ml of intact mitochondria, ruptured mitochondria and/or mitochondrial constituents selected from the group consisting of mitochondrial protein, mitochondrial nucleic acid, mitochondrial lipid and mitochondrial saccharide.

In certain embodiments, the treatment is selected from the group consisting of stopping hair follicle miniaturization, slowing hair follicle miniaturization, reversing hair follicle miniaturization, inducing elongation of hair follicles, enhancing elongation of hair follicles, inducing proliferation of cells within hair follicles, enhancing proliferation of cells within hair follicles, inducing elongation of hair fibers, enhancing elongation of hair fibers, enhancing thickness of hair fibers, altering the duration of hair follicle growth cycle phases and any combination thereof.

In certain embodiments, the treatment is inducing or enhancing elongation of hair follicles. In certain embodiments, the treatment is inducing or enhancing proliferation of cells within hair follicles.

In certain embodiments, the intact mitochondria, ruptured mitochondria and/or mitochondrial constituents are thawed.

In certain embodiments, the composition is formulated as a suspension, a colloid, a liquid, a lotion, a cream, an ointment, foam or a gel.

In certain embodiments, the intact mitochondria, ruptured mitochondria and/or mitochondrial constituents are derived from a cell or a tissue selected from the group consisting of placenta, placental cells grown in culture, blood cells, plant tissue, plant cells and plant cells grown in culture. In certain embodiments, the plant tissue is potato tissue or sprout tissue. In certain embodiments, the plant cells are potato cells or sprout cells.

The intact mitochondria, ruptured mitochondria and/or mitochondrial constituents according to the invention may be obtained by methods disclosed herein or by any other method known in the art. Commercially available mitochondria isolation kits include, for example Mitochondria Isolation Kit, MITOISO1 (Sigma-Aldrich), among others.

In a particular embodiment, the intact mitochondria, ruptured mitochondria and/or mitochondrial constituents are obtainable or obtained by a method comprising the steps: (1) Placenta was rinsed free of blood by using ice-cold IB buffer (isolation buffer: 200 mM sucrose, 1 mM EGTA and 10 mM Tris-MOPS)+0.2% BSA. (2) The placenta was minced into small pieces in 5 ml IB+0.2% BSA using scissors. (3) The suspension was transferred to a 10 ml glass potter and homogenized using a Dounce glass homogenizer by five complete up and down cycles. (4) The homogenate was transferred to a 15 ml tube and centrifuged at 600 g for 10 min at 4° C. (5) The supernatant was transferred to clean centrifuge tubes and the pellet was resuspended in IB buffer, and subjected to a second centrifugation step. (6) The supernatant from steps 4 and 5 was filtered through a 5 μm filter to remove any cells or large cell debris. (7) The supernatant was recovered and centrifuged at 7,000×g for 15 min (8) The mitochondrial pellet was washed in 10 ml ice cold IB buffer and mitochondria were recovered by centrifugation at 7,000×g for 15 min at 4° C. (9) The supernatant was discarded and the pellet resuspended, containing mitochondria in 200 μl of IB buffer.

In a particular embodiment, the intact mitochondria, ruptured mitochondria and/or mitochondrial constituents are obtainable or obtained by a method comprising the steps: (1) Placenta was rinsed free of blood by using ice-cold IB buffer (isolation buffer: 200 mM sucrose, 1 mM EGTA and 10 mM Tris-MOPS)+0.2% BSA. (2) The placenta was minced into small pieces in 5 ml IB+0.2% BSA using scissors. (3) The suspension was transferred to a 10 ml glass potter and homogenized using a Dounce glass homogenizer by five complete up and down cycles. (4) The homogenate was transferred to a 15 ml tube and centrifuged at 600 g for 10 min at 4° C. (5) The supernatant was transferred to clean centrifuge tubes and the pellet was resuspended in IB buffer, and subjected to a second centrifugation step. (6) The supernatant from steps 4 and 5 was filtered through a 5 μm filter to remove any cells or large cell debris. (7) The supernatant was recovered and centrifuged at 7,000×g for 15 min (8) The mitochondrial pellet was washed in 10 ml ice cold IB buffer and mitochondria were recovered by centrifugation at 7,000×g for 15 min at 4° C. (9) The supernatant was discarded and the pellet resuspended, containing mitochondria in 200 μl of IB buffer.

In a particular embodiment, the intact mitochondria, ruptured mitochondria and/or mitochondrial constituents are obtainable or obtained by a method comprising the steps: (1) potatoes were chilled over-night at 4° C., cut into small pieces and pulverized using a blender in the mannitol or sucrose containing buffer (at a 1:4 ratio of tissue:volume) for 30 seconds. (2) The mixtures were filtered through a cheese cloth and centrifuged at 600 g for 10 minutes at 4° C. (3) The supernatants were transferred to new tubes and centrifuged at 8000 g for 10 minutes. (4) The pellets of the mannitol/sucrose treated tissues were washed with 1 ml wash buffer (0.7 M Mannitol, 10 mM KPI pH 6.5) or isolation buffer, respectively, centrifuged at 8000 g for 10 minutes at 4° C. and re-suspended in 100 μl wash buffer/isolation buffer.

In a particular embodiment, the intact mitochondria, ruptured mitochondria and/or mitochondrial constituents are obtainable or obtained by a method comprising the steps: (1) Placenta was rinsed free of blood by using ice-cold M1 buffer (isolation buffer: 200 mM sucrose, 1 mM EGTA and 10 mM Tris-MOPS)+0.2% BSA. (2) The placenta was minced into small pieces in 5 ml M1+0.2% BSA using scissors. (3) The suspension was transferred to a 10 ml glass potter and homogenized using a Dounce glass homogenizer by five complete up and down cycles. (4) The homogenate was transferred to a 15 ml tube and centrifuged at 600 g for 10 min at 4° C. (5) The supernatant was transferred to clean centrifuge tubes and the pellet was resuspended in M1 buffer, and subjected to a second centrifugation step. (6) The supernatant from steps 4 and 5 was filtered through a 5 μm filter to remove any cells or large cell debris. (7) The supernatant was recovered and centrifuged at 7,000×g for 15 min (8) The mitochondrial pellet was washed in 10 ml ice cold M1 buffer and mitochondria were recovered by centrifugation at 7,000×g for 15 min at 4° C. (9) The supernatant was discarded and the pellet resuspended, containing mitochondria in 200 μl of M1 buffer.

In a particular embodiment, the intact mitochondria, ruptured mitochondria and/or mitochondrial constituents are obtainable or obtained by a method comprising the steps: (1) 400 gram of Vigna Radiata sprouts were washed and minced. (2) Homogenization in 2 L of Sucrose Buffer (250 mM Sucrose, 10 Mm Tris/HCl, 1 mM EDTA, pH 7.4). (3) Centrifugation at 600 g, 4° C. (4) Filter by 5 μm cutoff. (5) Centrifugation at 8000 g, 4° C. (6) Pellet wash and Centrifugation at 8000 g, 4° C.

In certain embodiments, the composition is administered by topical administration, oral administration, subcutaneous administration, intradermal administration, transdermal administration or systemic administration. Each possibility represents a separate embodiment of the present invention. In certain embodiments, the composition is administered by topical administration. In certain embodiments, the composition is administered by systemic administration. In certain embodiments, the composition is administered to a human scalp.

In certain embodiments, the subject is afflicted with a disease, disorder or condition which has a deleterious effect on hair vitality. In certain embodiments, the subject is afflicted with alopecia. In certain embodiments, the subject is afflicted with a mitochondrial disease. In certain embodiments, the mitochondrial disease is a deletion in mitochondrial DNA. In certain embodiments, the deletion in mitochondrial DNA is a 4977 bp deletion. In certain embodiments, the mitochondrial disease is Pearson syndrome. In certain embodiments, the subject is afflicted with cancer. In certain embodiments, the subject is treated by radiation or is to be treated by radiation. In certain embodiments, the subject is treated by chemotherapy or is to be treated by chemotherapy. In certain embodiments, the subject is afflicted with an autoimmune disorder. In certain embodiments, the autoimmune disorder is alopecia areata.

In certain embodiments, the subject is over 30, over 40, over 50 or over 60 years of age. Each possibility represents a separate embodiment of the present invention. In certain embodiments, the subject is a male.

In certain embodiments, the intact mitochondria, ruptured mitochondria and/or mitochondrial constituents are functional mitochondria. According to another embodiment, partially purified mitochondria are functional mitochondria. According to another embodiment, the mitochondria of the invention are isolated mitochondria. According to another embodiment, the mitochondria of the invention are intact mitochondria. According to another embodiment, the mitochondria of the invention are partially-functional. As used herein, “partially-functional mitochondria” refer to mitochondria lacking at least one functional property of mitochondria, such as, but not limited to, oxygen consumption. According to some embodiments, ruptured mitochondria are non-functional mitochondria. According to some embodiments, ruptured mitochondria are partially-functional mitochondria.

According to some embodiments, the term “functional mitochondria” refers to mitochondria that consume oxygen. According to another embodiment, functional mitochondria have an intact outer membrane. According to some embodiments, functional mitochondria are intact mitochondria. In another embodiment, functional mitochondria consume oxygen at an increasing rate over time. In another embodiment, the functionality of mitochondria is measured by oxygen consumption. In another embodiment, oxygen consumption of mitochondria may be measured by any method known in the art such as, but not limited to, the MitoXpress fluorescence probe (Luxcel). According to some embodiments, functional mitochondria are mitochondria which display an increase in the rate of oxygen consumption in the presence of ADP and a substrate such as, but not limited to, glutamate, malate or succinate. Each possibility represents a separate embodiment of the present invention. In another embodiment, functional mitochondria are mitochondria which produce ATP. In another embodiment, functional mitochondria are mitochondria capable of manufacturing their own RNAs and proteins and are self-reproducing structures. In another embodiment, functional mitochondria produce a mitochondrial ribosome and mitochondrial tRNA molecules.

As is known in the art, functional placental mitochondria participate in production of progesterone (see, for example, Tuckey R C, Placenta, 2005, 26(4):273-81). According to some embodiments, functional mitochondria are mitochondria which produce progesterone or pregnenolone. According to some embodiments, functional mitochondria are mitochondria which secrete progesterone. In a non-limiting example, mitochondria derived from placenta or placental cells grown in culture produce progesterone or pregnenolone. According to some embodiments, the mitochondria of the invention are derived from placenta or placental cells grown in culture and the mitochondria produce progesterone or pregnenolone. According to some embodiments, the production of progesterone or pregnenolone in the intact mitochondria of the invention is not impaired following a freeze-thaw cycle. According to some embodiments, the functionality of mitochondria is measured by measuring mitochondrial progesterone production or mitochondrial production of progesterone precursors such as, but not limited to, pregnenolone. Progesterone production may be measured by any assay known in the art such as, but not limited to, a radioimmunoassay (RIA).

As used herein, the term “partially purified mitochondria” refers to mitochondria separated from other cellular components, wherein the weight of the mitochondria constitutes between 20-80%, 30-80%, or 40-70% of the combined weight of the mitochondria and other sub-cellular fractions (as exemplified in: Hartwig et al., Proteomics, 2009, (9):3209-3214). According to another embodiment, partially purified mitochondria do not contain intact cells.

According to another embodiment, the weight of the mitochondria in partially purified mitochondria constitutes at least 20% of the combined weight of the mitochondria and other sub-cellular fractions. According to another embodiment, the weight of the mitochondria in partially purified mitochondria constitutes between 20%-40% of the combined weight of the mitochondria and other sub-cellular fractions. According to another embodiment, the weight of the mitochondria in partially purified mitochondria constitutes between 40%-80% of the combined weight of the mitochondria and other sub-cellular fractions. According to another embodiment, the weight of the mitochondria in partially purified mitochondria constitutes between 30%-70% of the combined weight of the mitochondria and other sub-cellular fractions. According to another embodiment, the weight of the mitochondria in partially purified mitochondria constitutes between 50%-70% of the combined weight of the mitochondria and other sub-cellular fractions. According to another embodiment, the weight of the mitochondria in partially purified mitochondria constitutes between 60%-70% of the combined weight of the mitochondria and other sub-cellular fractions. According to another embodiment, the weight of the mitochondria in partially purified mitochondria constitutes less than 80% of the combined weight of the mitochondria and other sub-cellular fractions.

As used herein, the term “mitochondrial proteins” refers to proteins which perform their function in the mitochondria, including mitochondrial proteins which are encoded by genomic DNA or mtDNA. As used herein, the term “cellular proteins” refers to all proteins which originate from the cells or tissue from which the mitochondria are produced.

As used herein, the term “isolated mitochondria” refers to mitochondria separated from other cellular components, wherein the weight of the mitochondria constitutes more than 80% of the combined weight of the mitochondria and other sub-cellular fractions. Preparation of isolated mitochondria may require changing buffer composition or additional washing steps, cleaning cycles, centrifugation cycles and sonication cycles which are not required in preparation of partially purified mitochondria. Without wishing to be bound by any theory or mechanism, such additional steps and cycles may harm the functionality of the isolated mitochondria.

According to another embodiment, the weight of the mitochondria in isolated mitochondria constitutes more than 90% of the combined weight of the mitochondria and other sub-cellular fractions. A non-limiting example of a method for obtaining isolated mitochondria is the MACS® technology (Miltenyi Biotec). Without wishing to be bound by any theory or mechanism, isolated mitochondria in which the weight of the mitochondria constitutes more than 95% of the combined weight of the mitochondria and other sub-cellular fractions are not functional mitochondria. According to another embodiment, isolated mitochondria do not contain intact cells. According to some embodiments, the mitochondria of the invention are isolated mitochondria.

As used herein, the term “intact mitochondria” refers to mitochondria comprising an outer membrane, an inner membrane, the cristae (formed by the inner membrane) and the matrix. In another embodiment, intact mitochondria comprise mitochondrial DNA. As used herein, the term “mitoplasts” refers to mitochondria devoid of outer membrane. According to another embodiment, intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show TMRM or TMRE staining have an intact mitochondrial outer membrane. According to some embodiments, intactness of a mitochondrial membrane is measured by assaying the presence of citrate synthase outside mitochondria. According to some embodiments, mitochondria that release citrate synthase have compromised mitochondrial intactness. According to some embodiments, intactness of a mitochondrial membrane is determined by measuring the mitochondrial rate of oxygen consumption coupled to presence of ADP. According to some embodiments, an increase in mitochondrial oxygen consumption in the presence of ADP is indicative of an intact mitochondrial membrane. According to some embodiments, intact mitochondria according to the invention are partially purified mitochondria. According to some embodiments, intact mitochondria according to the invention are isolated mitochondria. According to some embodiments, functional mitochondria are intact mitochondria.

As used herein, the term “a mitochondrial membrane” refers to a mitochondrial membrane selected from the group consisting of: the mitochondrial inner membrane, the mitochondrial outer membrane or a combination thereof.

As used herein, the term “ruptured mitochondria” refers to mitochondria in which the inner and outer mitochondrial membranes have been sheared (torn), perforated, punctured and the like. According to some embodiments, ruptured mitochondria are mitochondria that have been sheared to more than one piece/portion. It is to be understood that ruptured mitochondria are intact mitochondria that had been ruptured by the methods described herein or any other method known in the art.

According to some embodiments, ruptured mitochondria are mitochondria that released at least one mitochondrial constituent from the mitochondria. According to some embodiments, ruptured mitochondria are directed to mitochondria in which the inner and outer mitochondrial membranes have been sheared (torn), perforated, punctured and the like and which released at least one mitochondrial constituent. According to some embodiments, rupture of intact mitochondria results in release of at least one mitochondrial constituent. It is to be understood that, according to some embodiments, ruptured mitochondria that have released at least one mitochondrial constituent are administered together with the released constituent.

As used herein, the term “mitochondrial constituent” refers to any element comprised in mitochondria. According to some embodiments, a mitochondrial constituent is at least one element selected from the group consisting of: mitochondrial protein, mitochondrial nucleic acid, mitochondrial lipid, mitochondrial saccharide, mitochondrial structure, at least part of a mitochondrial matrix and a combination thereof. Each possibility represents a separate embodiment of the present invention.

As used herein, the term “mitochondrial structure” refers to structures and/or organelles present in mitochondria, such as, but not limited to, matrix granules, ATP-synthase particles, mitochondrial ribosomes and cristae. According to some embodiments, a mitochondrial constituent maintains at least one function of intact functional mitochondria. According to some embodiments, a mitochondrial constituent comprises a single type of mitochondrial protein, mitochondrial nucleic acid, mitochondrial lipid, mitochondrial structure or mitochondrial saccharide. Each possibility represents a separate embodiment of the present invention. According to some embodiments, a mitochondrial constituent comprises at least one functioning protein. According to some embodiments, a mitochondrial constituent comprises at least part of the mitochondrial matrix. According to some embodiments, a mitochondrial constituent comprises the entire mitochondrial matrix. According to some embodiments, a mitochondrial constituent comprises at least part of the mitochondrial matrix and at least part of the elements comprised therein, such as, but not limited to proteins, adenosine triphosphate (ATP) or ions. According to some embodiments, a mitochondrial constituent comprises at least part of the mitochondrial matrix and at least one of the following elements comprised therein: mitochondrial protein, mitochondrial nucleic acid, mitochondrial lipid, mitochondrial saccharide and a mitochondrial structure. Each possibility represents a separate embodiment of the present invention. As used herein, the term “mitochondrial matrix” refers to the viscous material within the mitochondrial inner membrane.

It is to be understood that mitochondrial constituents according to some embodiments of the present invention are elements secreted or released from mitochondria, such as, but not limited to mitochondrial proteins. According to some embodiments, mitochondrial constituents which are secreted or released from mitochondria may be retrieved by any method known in the art, such as, but not limited to, retrieving the mitochondrial constituents from a conditioned medium in which mitochondria have been incubated.

According to some embodiments, mitochondrial constituents may be obtained by any method known in the art for isolation of mitochondria fractions from cells, for example, the method carried out by using the Mitochondria isolation kit for culture cells from Thermo Fisher Scientific (Rockford, Ill., USA). According to some embodiments, mitochondrial fractions or constituents are produced as a byproduct of mitochondria isolation or partial purification. Each possibility represents a separate embodiment of the present invention.

According to some embodiments, a mitochondrial constituent according to the present invention comprises progesterone. According to some embodiments, progesterone is released from ruptured mitochondria. According to some embodiments, the composition of the invention comprises ruptured mitochondria and progesterone released from the mitochondria. In a non-limiting example, ruptured mitochondria and/or mitochondrial constituents of mitochondria derived from placenta or placental cells grown in culture may comprise progesterone. According to some embodiments, ruptured mitochondria and/or mitochondrial constituents comprising progesterone inhibit enzymes of the 5α-reductase family. Each possibility represents a separate embodiment of the present invention. As used herein, “5α-reductase family” is a family of enzymes involved in steroid metabolism, mainly in conversion of testosterone to 5α-dihydrotestosterone (DHT). As known in the art, DHT affects hair follicle miniaturization and alopecia. Therefore, inhibition of enzymes of the 5α-reductase family, thus reducing DHT levels, may help stop hair loss and even induce hair re-growth. Without wishing to be bound by any theory or mechanism, mitochondrial constituents comprising progesterone may be efficient for treating hair loss/alopecia due to inhibition of 5α-reductase, thus preventing conversion of testosterone to 5α-dihydrotestosterone (DHT).

It is to be understood that ruptured mitochondria and/or mitochondrial constituents according to some embodiments of the present invention are obtained from intact and/or isolated and/or partially purified mitochondria. It is to be further understood that mitochondrial constituents according to embodiments of the present invention are obtained from intact mitochondria through any method known in the art. According to some embodiments, the mitochondrial constituents of the invention are obtained by transferring the intact mitochondria from a hypertonic solution to a hypotonic solution. According to some embodiments, transferring intact mitochondria from a hypertonic to a hypotonic solution results in release of at least one mitochondrial constituent. Each possibility represents a separate embodiment of the present invention.

According to some embodiments, the present invention provides a method for treatment of hair follicles, the method comprising administering to a subject in need thereof an effective amount of a composition comprising at least one mitochondrial constituent. According to some embodiments, the present invention provides a method for treatment of alopecia, the method comprising administering to a subject afflicted with alopecia an effective amount of a composition comprising at least one mitochondrial constituent.

As used herein, the terms “hypotonic”, “isotonic” and “hypertonic” relate to a concentration relative to the solute concentration inside intact mitochondria.

According to other embodiments, ruptured mitochondria are obtained by exposing intact mitochondria to a hypotonic solution, such as, but not limited to, a hypotonic phosphate-buffered saline (PBS) solution. Without wishing to be bound by any theory or mechanism, exposing intact mitochondria to a hypotonic solution results in explosion or perforation of the mitochondria, thus obtaining ruptured mitochondria, possibly releasing mitochondrial constituents such as, but not limited to, at least part of the mitochondrial matrix.

According to some embodiments, ruptured mitochondria are obtained by transferring mitochondria from a hypertonic solution to a hypotonic solution. Without wishing to be bound by any theory or mechanism, transferring intact mitochondria from a hypertonic solution to a hypotonic solution results in explosion, rupture or perforation of the mitochondria, thus obtaining ruptured mitochondria, possibly releasing mitochondrial constituents such as, but not limited to, at least part of the mitochondrial matrix. In a non-limiting example, explosion, rupture or perforation of intact mitochondria may result in release of mitochondrial proteins such as citrate synthase. According to some embodiments, release of citrate synthase is used as an indication of ruptured mitochondria. According to some embodiments, mitochondrial constituents according to the present invention are released from intact mitochondria by increasing the osmotic pressure within the intact mitochondria. Without wishing to be bound by any theory or mechanism, increasing the osmotic pressure within intact mitochondria such that mitochondrial membranes are perforated and/or torn results in ruptured mitochondria and possibly in release of mitochondrial constituents according to the present invention.

According to some embodiments, a composition comprising intact mitochondria according to the present invention is formulated as a hypertonic solution. According to some embodiments, the composition of the invention comprises a hypertonic solution. According to some embodiments, a hypertonic solution according to the present invention comprises a saccharide. As used herein the term “saccharide” may refer to a saccharide, an oligosaccharide or a polysaccharide. Each possibility represents a separate embodiment of the present invention. According to some embodiments, the saccharide is sucrose. According to some embodiments, the concentration of the saccharide in the hypertonic solution according to the present invention is similar to the concentration of the saccharide in the isolation buffer. According to some embodiments, a sufficient saccharide concentration which acts to preserve mitochondrial function is sufficient for preserving mitochondria intact. According to some embodiments, the isolation buffer is hypertonic. According to other embodiments, the saccharide concentration in the hypertonic solution, according to the present invention, is a sufficient saccharide concentration for preserving mitochondria intact. According to some embodiments, the composition of the invention further comprises a sufficient saccharide concentration for preserving mitochondria intact.

According to another embodiment, a sufficient saccharide concentration for preserving mitochondria intact is a concentration of between 100 mM-400 mM, preferably between 100 mM-250 mM, most preferably between 200 mM-250 mM. Each possibility represents a separate embodiment of the present invention. According to another embodiment, a sufficient saccharide concentration for preserving mitochondria intact is between 100 mM-150 mM. According to another embodiment, a sufficient saccharide concentration for preserving mitochondria intact is between 150 mM-200 mM. According to another embodiment, a sufficient saccharide concentration for preserving mitochondria intact is between 100 mM-200 mM. According to another embodiment, a sufficient saccharide concentration for preserving mitochondria intact is between 100 mM-400 mM. According to another embodiment, a sufficient saccharide concentration for preserving mitochondria intact is between 150 mM-400 mM. According to another embodiment, a sufficient saccharide concentration for preserving mitochondria intact is between 200 mM-400 mM. According to another embodiment, a sufficient saccharide concentration for preserving mitochondria intact is at least 100 mM. Without wishing to be bound by any theory or mechanism of action, a saccharide concentration below 100 mM may not be sufficient to preserve mitochondria intact. According to some embodiments, a saccharide concentration above 100 mM is hypertonic.

According to some embodiments, a composition comprising ruptured mitochondria according to the present invention is formulated as a hypotonic solution. According to some embodiments, the composition of the invention comprises a hypotonic solution. A non-limiting example of a hypotonic solution is Phosphate Buffered Saline (PBS). According to some embodiments, mitochondria in PBS are ruptured mitochondria. According to other embodiments, mitochondria in isolation buffer are intact mitochondria. According to some embodiments, mitochondria in an isolation buffer comprising a saccharide concentration sufficient for preserving mitochondria intact are intact mitochondria.

According to some embodiments, the intact mitochondria of the invention are exposed to an ion-exchanger inhibitor. According to some embodiments, the intact mitochondria of the invention are reduced in size by exposure to an ion-exchanger inhibitor. According to another embodiment, the intact mitochondria of the invention were reduced in size by exposure to an ion-exchanger inhibitor. According to some embodiments, the intact mitochondria of the invention are exposed to the ion-exchanger inhibitor following partial purification or isolation. Each possibility represents a separate embodiment of the present invention. According to some embodiments, the intact mitochondria of the invention are exposed to the ion-exchanger inhibitor during partial purification or isolation. Each possibility represents a separate embodiment of the present invention. According to other embodiments, the cells or tissue from which the intact mitochondria of the invention are derived are exposed to the ion-exchanger inhibitor prior to partial purification or isolation of the mitochondria. Each possibility represents a separate embodiment of the present invention. According to another embodiment, the ion-exchanger inhibitor is CGP37157. As used herein, the terms “CGP” and “CGP37157” are used interchangeably. Without wishing to be bound by any theory or mechanism, agents blocking the mitochondrial Na⁺/Ca²⁺ exchanger, such as, CGP37157 may induce mitochondrial fission, increase mitochondrial ATP production and reduce mitochondrial size. Mitochondrial fission refers to spontaneous fission or fission induced by appropriate agents such as CGP37157. According to another embodiment, the final composition of the invention is devoid of free ion-exchanger inhibitor. As used herein, a composition devoid of ion-exchanger inhibitor refers to a composition devoid of ion-exchanger inhibitor which is not bound to the mitochondria of the invention. According to some embodiments, the composition of the invention comprises an ion-exchanger inhibitor bound to the mitochondria of the invention. According to some embodiments, a composition devoid of ion-exchanger inhibitor comprises an ion-exchanger inhibitor at a concentration of less than 1 μM of, preferably less than 0.5 μM, most preferably less than 0.1 μM.

According to another embodiment, the mitochondria of the invention are derived from a different subject than the subject to whom they are administered. According to another embodiment, the mitochondria of the invention are from a source selected from allogeneic and xenogeneic. Each possibility represents a separate embodiment of the present invention. According to another embodiment, the mitochondria of the invention are derived from a cell or tissue from a source selected from allogeneic and xenogeneic. Each possibility represents a separate embodiment of the present invention.

As used herein, mitochondria of an allogeneic source refer to mitochondria derived from a different subject than the subject to be treated from the same species. As used herein, mitochondria of a xenogeneic source refer to mitochondria derived from a different subject than the subject to be treated from a different species. According to some embodiment, a xenogeneic source is a plant source.

According to another embodiment, the mitochondria of the invention are derived from a mammalian subject. According to another embodiment, the mammalian subject is a human subject. According to another embodiment, the mitochondria of the invention are derived from a mammalian cell. According to another embodiment, the mammalian cell is a human cell. According to another embodiment, the mitochondria of the invention are derived from cells in culture. According to another embodiment, the mitochondria of the invention are derived from a tissue.

According to some embodiments, the mitochondria of the invention are derived from plant tissue, plant cells or plant cells grown in culture. Each possibility represents a separate embodiment of the present invention. According to some embodiments, deriving mitochondria from plant tissue, plant cells or plant cells grown in culture according to the present invention refers to deriving mitochondria from plant protoplasts. Each possibility represents a separate embodiment of the present invention. Plant mitochondria according to the present invention may be derived from any plant species, plant organ, plant cells or plant cells grown in culture known in the art to comprise mitochondria. Each possibility represents a separate embodiment of the present invention. In non-limiting examples, plant mitochondria according to the invention may be derived from storage organs (such as potato, sugar or beet), green leaves (such as tobacco, pea or petunia) or etiolated seedlings (such as wheat, maize or mung bean). According to some embodiments, the mitochondria of the invention are derived from potato. According to some embodiments, the mitochondria of the invention are derived from algae, such as but not limited to, dunaliella. According to embodiments, the mitochondria of the invention are obtained from an animal subject, preferably a mammalian subject, most preferably a human subject or human cells grown in culture. Each possibility represents a separate embodiment of the present invention. According to some embodiments, the mitochondria of the invention are obtained from cells lacking a cell wall, preferably mammalian cells, most preferably human cells. Each possibility represents a separate embodiment of the present invention.

According to another embodiment, the mitochondria of the invention are derived from a cell or a tissue selected from the group consisting of: human placenta, human placental cells grown in culture and human blood cells. Each possibility represents a separate embodiment of the present invention. According to another embodiment, the mitochondria of the invention are derived from a cell or a tissue selected from the group consisting of: placenta, placental cells grown in culture and blood cells. Each possibility represents a separate embodiment of the present invention. According to another embodiment, the mitochondria of the invention are derived from a cell or a tissue selected from the group consisting of: human placenta, human placental cells grown in culture, human blood cells, plant tissue, plant cells and plant cells grown in culture. Each possibility represents a separate embodiment of the present invention. According to another embodiment, the mitochondria of the invention are derived from a cell or a tissue selected from the group consisting of: placenta, placental cells grown in culture, blood cells, plant tissue, plant cells and plant cells grown in culture. Each possibility represents a separate embodiment of the present invention.

According to some embodiments, the mitochondrial constituent according to the present invention is produced from mitochondria derived from a cell or a tissue selected from the group consisting of: placenta, placental cells grown in culture and blood cells. Each possibility represents a separate embodiment of the present invention. According to some embodiments, the mitochondrial constituent according to the present invention is produced from mitochondria derived from a cell or a tissue selected from the group consisting of: human placenta, human placental cells grown in culture and human blood cells. Each possibility represents a separate embodiment of the present invention. According to some embodiments, the mitochondrial constituent according to the present invention is produced from mitochondria derived from a cell or a tissue selected from the group consisting of: placenta, placental cells grown in culture, blood cells, plant tissue, plant cells and plant cells grown in culture. Each possibility represents a separate embodiment of the present invention. According to some embodiments, the mitochondrial constituent according to the present invention is produced from mitochondria derived from a cell or a tissue selected from the group consisting of: human placenta, human placental cells grown in culture and human blood cells. Each possibility represents a separate embodiment of the present invention. According to some embodiments, the mitochondrial constituent according to the present invention is produced from mitochondria derived from a cell or a tissue selected from the group consisting of: human placenta, human placental cells grown in culture, human blood cells, plant tissue, plant cells and plant cells grown in culture. Each possibility represents a separate embodiment of the present invention.

As used herein. the phrases “cells grown in culture” or “a tissue grown in culture” refers to a multitude of cells or a tissue, respectively, grown in a liquid, semi-solid or solid medium, outside of the organism from which the cells or tissue derive. According to some embodiments, cells grown in culture are cells grown in bioreactors. According to a non-limiting example, cells may be grown in a bioreactor (such as, but not limited to the bioreactor disclosed in WO 2008/152640), followed by isolation or partial purification of mitochondria from cells.

According to another embodiment, the mitochondria of the invention have undergone a freeze-thaw cycle. According to some embodiments, the intact mitochondria of the invention have undergone a freeze-thaw cycle. Without wishing to be bound by any theory or mechanism, intact mitochondria that have undergone a freeze-thaw cycle demonstrate at least comparable oxygen consumption rate following thawing, as compared to control intact mitochondria that have not undergone a freeze-thaw cycle. Thus, intact mitochondria that have undergone a freeze-thaw cycle are at least as functional as control mitochondria that have not undergone a freeze-thaw cycle.

As used herein, the term “freeze-thaw cycle” refers to freezing of the mitochondria of the invention to a temperature below 0° C., maintaining the mitochondria in a temperature below 0° C. for a defined period of time and thawing the mitochondria to room temperature or body temperature or any temperature above 0° C. which enables administration according to the methods of the invention. Each possibility represents a separate embodiment of the present invention. The term “room temperature”, as used herein refers to a temperature of between 18° C. and 25° C. The term “body temperature”, as used herein, refers to a temperature of between 35.5° C. and 37.5° C., preferably 37° C.

In another embodiment, the mitochondria that have undergone a freeze-thaw cycle were frozen at a temperature of at least −196° C. In another embodiment, the mitochondria that have undergone a freeze-thaw cycle were frozen at a temperature of at least −70° C. In another embodiment, the mitochondria that have undergone a freeze-thaw cycle were frozen at a temperature of at least −20° C. In another embodiment, the mitochondria that have undergone a freeze-thaw cycle were frozen at a temperature of at least −4° C. In another embodiment, the mitochondria that have undergone a freeze-thaw cycle were frozen at a temperature of at least 0° C. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures. In a non-limiting example, flash-freezing may include freezing using liquid nitrogen.

In another embodiment, the mitochondria that underwent a freeze-thaw cycle were frozen for at least 30 minutes prior to thawing. According to another embodiment, the freeze-thaw cycle comprises freezing the mitochondria for at least 30, 60, 90, 120, 180, 210 minutes prior to thawing. Each possibility represents a separate embodiment of the present invention. In another embodiment, the mitochondria that have undergone a freeze-thaw cycle were frozen for at least 1, 2, 3, 4, 5, 6, 7, 8, 9, 10, 24, 48, 72, 96, 120 hours prior to thawing. Each freezing time presents a separate embodiment of the present invention. In another embodiment, the mitochondria that have undergone a freeze-thaw cycle were frozen for at least 4, 5, 6, 7, 30, 60, 120, 365 days prior to thawing. Each freezing time presents a separate embodiment of the present invention. According to another embodiment, the freeze-thaw cycle comprises freezing the mitochondria for at least 1, 2, 3 weeks prior to thawing. Each possibility represents a separate embodiment of the present invention. According to another embodiment, the freeze-thaw cycle comprises freezing the mitochondria for at least 1, 2, 3, 4, 5, 6 months prior to thawing. Each possibility represents a separate embodiment of the present invention.

In another embodiment, the mitochondria that have undergone a freeze-thaw cycle were frozen at −70° C. for at least 30 minutes prior to thawing. Without wishing to be bound by any theory or mechanism, the possibility to freeze mitochondria and thaw them after a long period enables easy storage and use of the mitochondria with reproducible results even after a long period of storage. According to some embodiments, ruptured mitochondria according to the present invention are prepared/produced from intact mitochondria that have undergone a freeze-thaw cycle.

According to another embodiment, thawing is at room temperature. In another embodiment, thawing is at body temperature. According to another embodiment, thawing is at a temperature which enables administration according to the methods of the invention. According to another embodiment, thawing is performed gradually.

As used herein, the term “isolation buffer” refers to a buffer in which the mitochondria of the invention have been partially purified or isolated. Each possibility represents a separate embodiment of the present invention. It is to be understood that intact mitochondria according to the invention are isolated or partially purified in isolation buffer, while ruptured mitochondria are produced from isolated/partially purified intact mitochondria by methods described herein or any other method known in the art. In a non-limiting example, the isolation buffer comprises 200 mM sucrose, 10 mM Tris-MOPS and 1 mM EGTA. According to some embodiments, BSA (Bovine Serum Albumin) is added to the isolation buffer during partial purification or isolation. Each possibility represents a separate embodiment of the present invention. According to some embodiments, 0.2% BSA is added to the isolation buffer during partial purification or isolation. Each possibility represents a separate embodiment of the present invention. According to some embodiments, HSA (Human Serum Albumin) is added to the isolation buffer during partial purification or isolation. Each possibility represents a separate embodiment of the present invention. According to some embodiments, 0.2% HSA is added to the isolation buffer during partial purification or isolation. Each possibility represents a separate embodiment of the present invention. According to other embodiment, HSA or BSA is washed away from the mitochondria of the invention following partial purification or isolation. Each possibility represents a separate embodiment of the present invention. Without wishing to be bound by any mechanism or theory, freezing mitochondria within the isolation buffer saves time and isolation steps, as there is no need to replace the isolation buffer with a freezing buffer prior to freezing or to replace the freezing buffer upon thawing.

According to another embodiment, the mitochondria that underwent a freeze-thaw cycle were frozen within a freezing buffer. According to another embodiment, the intact mitochondria that underwent a freeze-thaw cycle were frozen within the isolation buffer. According to another embodiment, the intact mitochondria that underwent a freeze-thaw cycle were frozen within a buffer comprising the same constituents as the isolation buffer.

According to another embodiment, the freezing buffer comprises a cryoprotectant. According to some embodiments, the cryoprotectant is a saccharide, an oligosaccharide or a polysaccharide. Each possibility represents a separate embodiment of the present invention. According to another embodiment, the saccharide concentration in the freezing buffer is a sufficient saccharide concentration which acts to preserve mitochondrial function. According to another embodiment, the isolation buffer comprises a saccharide. According to another embodiment, the saccharide concentration in the isolation buffer is a sufficient saccharide concentration which acts to preserve mitochondrial function. According to another embodiment, the saccharide concentration in the isolation buffer is a sufficient saccharide concentration which acts to keep mitochondria intact. According to another embodiment, the saccharide concentration in the freezing buffer is a sufficient saccharide concentration which acts to keep mitochondria intact. According to another embodiment, the saccharide is sucrose. Without wishing to be bound by any theory or mechanism, intact mitochondria that have been frozen within a freezing buffer or isolation buffer comprising sucrose demonstrate at least comparable oxygen consumption rate following thawing, as compared to control mitochondria that have not undergone a freeze-thaw cycle or that have been frozen within a freezing buffer or isolation buffer without sucrose.

According to some embodiments, ruptured mitochondria underwent a freeze-thaw cycle. According to some embodiments, a mitochondrial constituent according to the invention underwent a freeze-thaw cycle. According to some embodiments, the ruptured mitochondria that underwent a freeze-thaw cycle were frozen within a freezing buffer. According to some embodiments, the mitochondrial constituent that underwent a freeze-thaw cycle was frozen within a freezing buffer. According to some embodiments, the ruptured mitochondria that underwent a freeze-thaw cycle were frozen within a hypotonic solution, such as, but not limited to PBS. According to some embodiments, the mitochondrial constituent that underwent a freeze-thaw cycle was frozen within a hypotonic solution, such as, but not limited to PBS.

According to some embodiments, the ruptured mitochondria that underwent a freeze-thaw cycle were frozen within the isolation buffer. According to another embodiment, the ruptured mitochondria that underwent a freeze-thaw cycle were frozen within a buffer comprising the same constituents as the isolation buffer. According to some embodiments, the mitochondrial constituent that underwent a freeze-thaw cycle was frozen within the isolation buffer. According to another embodiment, the mitochondrial constituent that underwent a freeze-thaw cycle was frozen within a buffer comprising the same constituents as the isolation buffer.

Any suitable route of administration to a subject may be used according to the methods of the present invention, including but not limited to local routes. According to some embodiments, administering is administering locally. According to some embodiments, the composition is formulated for local administration.

According to another embodiment, administration is through a parenteral route. According to some embodiments, preparations of the composition of the invention for parenteral administration include sterile aqueous or non-aqueous solutions, suspensions, or emulsions, each representing a separate embodiment of the present invention. Non-limiting examples of non-aqueous solvents or vehicles are propylene glycol, polyethylene glycol, vegetable oils such as olive oil and corn oil, gelatin, and injectable organic esters such as ethyl oleate.

According to some embodiments, parenteral administration is administration intradermally or subcutaneously. Each of the abovementioned administration routes represents a separate embodiment of the present invention. According to another embodiment, parenteral administration is performed by bolus injection. The preferred mode of administration will depend upon the particular indication being treated and will be apparent to one of skill in the art.

According to another embodiment, local administration of the composition is through injection. According to some embodiments, injection according to the methods of the invention is injection into the scalp. For administration through injection, the composition may be formulated in an aqueous solution, for example in a physiologically compatible buffer including but not limited to Hank's solution, Ringer's solution, or physiological salt buffer. Formulations for injection may be presented in unit dosage forms, for example, in ampoules, or in multi-dose containers with, optionally, an added preservative.

Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate or triglycerides

According to some embodiments, administering is administering topically. According to some embodiments, the composition is formulated for topical administration. The term “topical administration”, as used herein, refers to administration to body surfaces. Non-limiting examples of formulations for topical use include cream, ointment, lotion, gel, foam, suspension, aqueous or co-solvent solutions, salve and spray-able liquid forms. Other suitable topical product forms suitable for use with the methods of the present invention include, for example, emulsion, mousse, lotion, solution and serum.

Compositions formulated for topical administration may comprise, without limitation, non-washable (water-in-oil) creams or washable (oil-in-water) creams, ointments, lotions, gels, suspensions, aqueous or cosolvent solutions, salves, emulsions, wound dressings, coated bandages or other polymer coverings, sprays, aerosols, liposomes and any other acceptable carrier suitable for administration of the drug topically.

As is well known in the art the physico-chemical characteristics of the composition may be manipulated by addition a variety of excipients, including but not limited to thickeners, gelling agents, wetting agents, flocculating agents, suspending agents and the like. These optional excipients will determine the physical characteristics of the resultant formulations such that the application may be more pleasant or convenient. It will be recognized by the skilled artisan that the excipients selected, should preferably enhance and in any case must not interfere with the storage stability of the formulations.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

For example, a cream formulation may comprise in addition to the active compound: (a) a hydrophobic component; (b) a hydrophilic aqueous component; and (c) at least one emulsifying agent. The hydrophobic component of the cream is exemplified by the group consisting of mineral oil, yellow soft paraffin (Vaseline), white soft paraffin (Vaseline), paraffin (hard paraffin), paraffin oil heavy, hydrous wool fat (hydrous lanolin), wool fat (lanolin), wool alcohol (lanolin alcohol), petrolatum and lanolin alcohols, beeswax, cetyl alcohol, almond oil, arachis oil, castor oil, hydrogenated castor oil wax, cottonseed oil, ethyl oleate, olive oil, sesame oil, and mixtures thereof. The hydrophilic aqueous component of the cream is exemplified by water alone, propylene glycol or alternatively any pharmaceutically acceptable buffer or solution. Emulsifying agents are added to the cream in order to stabilize the cream and to prevent the coalescence of the droplets. The emulsifying agent reduces the surface tension and forms a stable, coherent interfacial film. A suitable emulsifying agent may be exemplified by but not limited to the group consisting of cholesterol, cetostearyl alcohol, wool fat (lanolin), wool alcohol (lanolin alcohol), hydrous wool fat (hydrous lanolin), and mixtures thereof.

A topical suspension, for example, may comprise in addition to the active compound: (a) an aqueous medium; and (b) suspending agents or thickeners. Optionally additional excipients are added. Suitable suspending agent or thickeners may be exemplified by but not limited to the group consisting of cellulose derivatives like methylcellulose, hydroxyethylcellulose and hydroxypropyl cellulose, alginic acid and its derivatives, xanthan gum, guar gum, gum arabic, tragacanth, gelatin, acacia, bentonite, starch, microcrystalline cellulose, povidone and mixture thereof. The aqueous suspensions may optionally contain additional excipients e.g. wetting agents, flocculating agents, thickeners, and the like. Suitable wetting agents are exemplified by but not limited to the group consisting of glycerol polyethylene glycol, polypropylene glycol and mixtures thereof, and surfactants. The concentration of the wetting agents in the suspension should be selected to achieve optimum dispersion of the pharmaceutical powders within the suspension with the lowest feasible concentration of the wetting agent. Suitable flocculating agents are exemplified by but not limited to the group consisting of electrolytes, surfactants, and polymers. The suspending agents, wetting agents and flocculating agents are provided in amounts that are effective to form a stable suspension of the pharmaceutically effective agent. As used herein, the term “active compound” refers to mitochondria, mitochondrial constituents or a combination thereof.

Topical gel formulation, for example, may comprise in addition to the active compound, at least one gelling agent and an acid compound. Suitable gelling agents may be exemplified by but not limited to the group consisting of hydrophilic polymers, natural and synthetic gums, crosslinked proteins and mixture thereof. The polymers may comprise for example hydroxyethylcellulose, hydroxyethyl methylcellulose, methyl cellulose, hydroxypropylcellulose, hydroxypropyl methylcellulose, carboxymethyl cellulose, and similar derivatives of amylose, dextran, chitosan, pullulan, and other polysaccharides; Crosslinked proteins such as albumin, gelatin and collagen; acrylic based polymer gels such as Carbopol, Eudragit and hydroxyethyl methacrylate based gel polymers, polyurethane based gels and mixtures thereof.

Topical compositions of the present invention may additionally be formulated as a solution. Such a solution comprises, in addition to the active compound, at least one co-solvent exemplified but not limited to the group consisting of water, buffered solutions, organic solvents such as ethyl alcohol, isopropyl alcohol, propylene glycol, polyethylene glycol, glycerin, glycoforol, Cremophor, ethyl lactate, methyl lactate, N-methylpyrrolidone, ethoxylated tocopherol and mixtures thereof.

According to some embodiments, the composition of the invention is administered topically. According to some embodiments, the composition of the invention is administered topically to the scalp of a subject in need thereof. According to some embodiments, topical administration according to the methods of the present invention is administration to the hair follicles. According to another embodiment, topical administration according to the methods of the invention is administration wherein the composition of the invention is formulated as a shampoo, ointment, spray, gel or liquid. Each possibility represents a separate embodiment of the present invention.

According to some embodiments, the composition of the invention is formulated as a cosmetic formulation. According to some embodiments, the composition of the invention is formulated as a cosmetic formulation for topical administration. Non-limiting examples of cosmetic formulations include, but are not limited to a tonic, a lotion, a cream, an ointment, a gel, a shampoo, a spray (aerosol or mist), a conditioner, a hairdye, a rinse and the like. According to some embodiments, the composition of the invention comprises a stimulant of blood circulation, such as, but not limited to cepharanthin, carpronium chloride, sialid extract and garlic extract. According to some embodiments, the composition of the invention comprises topical stimulators such as, but not limited to, capsicum tincture, cantharis tincture, ginger tincture, nonylic acid vanillylamide and the like. According to some embodiments, the composition of the invention comprises cosmetic materials such as, but not limited to, fragrance, a moisturizing element, a dye, a hair softening element, a hair conditioning element and the like.

According to some embodiments, administering to a subject in need thereof is by a route selected from the group consisting of: topical, subcutaneous, intradermal and through direct injection into a tissue or an organ. According to some embodiments, administration through direct injection according to the methods of the present invention is injection into the scalp of a subject in need thereof.

According to some embodiments, treatment of hair follicles according to the methods of the invention is by administration of a composition comprising ruptured mitochondria. According to some embodiments, treating a subject afflicted with alopecia according to the methods of the invention is by administration of a composition comprising ruptured mitochondria. According to some embodiments, treatment of hair follicles according to the methods of the invention is by administration of a composition comprising ruptured mitochondria and at least one mitochondrial constituent released from the ruptured mitochondria. According to some embodiments, treating a subject afflicted with alopecia according to the methods of the invention is by administration of a composition comprising ruptured mitochondria and at least one mitochondrial constituent released from the ruptured mitochondria. According to some embodiments, treatment of hair follicles according to the methods of the invention is by administration of at least one mitochondrial constituent. According to some embodiments, treating a subject afflicted with alopecia according to the methods of the invention is by administration of at least one mitochondrial constituent.

According to some embodiments, treatment of hair follicles according to the methods of the invention is by administration of intact mitochondria. According to some embodiments, treating a subject afflicted with alopecia according to the methods of the invention is by administration of intact mitochondria. According to some embodiments, treatment of hair follicles according to the methods of the invention is by administration of partially purified mitochondria. According to some embodiments, treating a subject afflicted with alopecia according to the methods of the invention is by administration of partially purified mitochondria. According to some embodiments, treatment of hair follicles according to the methods of the invention is by administration of isolated mitochondria. According to some embodiments, treating a subject afflicted with alopecia according to the methods of the invention is by administration of isolated mitochondria.

According to some embodiments, treatment of hair follicles according to the methods of the invention is by administration of intact mitochondria and/or ruptured mitochondria and/or at least one mitochondrial constituent. Each possibility represents a separate embodiment of the present invention. According to some embodiments, treating a subject afflicted with alopecia according to the methods of the invention is by administration of intact mitochondria and/or ruptured mitochondria and/or at least one mitochondrial constituent. Each possibility represents a separate embodiment of the present invention.

As used herein, the term “hair follicle” refers to a hair producing organ situated within the skin and comprising dermal papilla cells, follicular matrix, root sheath and a hair fiber. As used herein, elongation of hair follicle relates to elongation of any part of the hair follicle such as, but not limited to, the root sheath. As used herein, the term “hair follicle growth cycle” relates to a growth cycle comprising essentially the anagen, catagen and telogen phases, as disclosed in the background hereinabove.

As used herein, the terms “treating”, “treating according to the present invention”, “treatment”, “the treatment according to the present invention”, “treating hair follicles”, “treating hair follicle” and “treating a hair follicle” are interchangeable and relate to at least one treatment selected from the group consisting of: inducing elongation of hair follicles, enhancing elongation of hair follicles, inducing elongation of hair fibers, enhancing elongation of hair fibers, enhancing thickness of hair fibers, inducing proliferation of cells within hair follicles, enhancing proliferation of cells within hair follicles, stopping hair follicle miniaturization, slowing hair follicle miniaturization, reversing hair follicle miniaturization, altering the duration of hair follicle growth cycle phases and a combination thereof. Each possibility represents a separate embodiment of the present invention. According to some embodiments, altering the duration of hair follicle growth cycle phases relates to elongation of the anagen phase.

According to some embodiments, treatment of a hair follicle relates to inducing elongation of a hair follicle. According to some embodiments, treatment of a hair follicle relates to enhancing elongation of a hair follicle. According to some embodiments, treatment of a hair follicle relates to inducing or enhancing elongation of a hair follicle. Each possibility represents a separate embodiment of the present invention. According to some embodiments, treatment of a hair follicle relates to inducing elongation of a hair fibre. According to some embodiments, treatment of a hair follicle relates to enhancing elongation of a hair fiber. According to some embodiments, treatment of a hair follicle comprises elongation of the hair fiber. As used herein, the terms “cells within hair follicles” and “cells within the hair follicle” are interchangeable and relate to cells comprised in the hair follicle, such as, but not limited to dermal papilla cells. According to some embodiments, treatment of hair follicles comprises inducing proliferation of dermal papilla cells. According to some embodiments, treatment of hair follicles comprises enhancing proliferation of dermal papilla cells. According to some embodiments, treatment according to the present invention comprises affecting the function of cells within hair follicles, such as, but not limited to, dermal papilla cells.

Without wishing to be bound by any theory or mechanism, treatment of hair follicles according to the present invention may result in at least one of the following: hair growth, prevention of hair shedding, slowing down of hair shedding, increasing rate of hair growth, increasing hair quantity, increasing hair thickness, increasing hair strength or a combination thereof.

As used herein, a subject in need thereof is a subject afflicted with a disease or disorder which would benefit from treatment of hair follicles. According to some embodiments, a disease or disorder which would benefit from treatment of hair follicles is alopecia. According to some embodiments, a disease or disorder which would benefit from treatment of hair follicles is any disease or disorder known in the art which may cause hair loss, hair growth impairment, hair thinning, delay of hair growth rate and a combination thereof. Each possibility represents a separate embodiment of the present invention. According to other embodiments, a disease or disorder which would benefit from treatment of hair follicles is any disease or disorder known in the art to cause side effects selected from the group consisting of: hair loss, hair growth impairment, hair thinning, delay of hair growth rate and a combination thereof. Each possibility represents a separate embodiment of the present invention. According to other embodiments, a disease or disorder which would benefit from treatment of hair follicles is any disease or disorder known in the art to have treatment methods which induce hair loss, hair growth impairment, hair thinning, delay of hair growth rate and a combination thereof. Each possibility represents a separate embodiment of the present invention. A non-limiting example of a treatment method which may induce hair impairment is chemotherapy for treatment of various types of cancer.

As used herein, the term “alopecia” refers to loss of hair from the head or body, including, but not limited to, androgenic alopecia commonly referred to as male-pattern baldness.

The term “effective amount”, as used herein, refers to an amount of the composition of the invention sufficient for achieving a desired effect in hair follicles according to the present invention. According to some embodiments, an effective amount is an amount of the composition of the invention which results in amelioration of alopecia.

According to some embodiments, the methods of the invention further comprise administration of at least one hair-growth inducing agent. As used herein, a hair-growth inducing agent, according to some embodiments, is any substance or composition known in the art to have an effect on hair-growth such as, but not limited to, induction of hair growth, prevention of hair shedding, slowing down of hair shedding, increasing rate of hair growth, increasing hair quantity, increasing hair thickness, increasing hair strength or a combination thereof. Each possibility represents a separate embodiment of the present invention. According to some embodiments, the hair-growth inducing agent is selected from the group consisting of: Finasteride, Dutasteride, Minoxidil, Kopexil, oxidized coenzyme Q, reduced coenzyme Q, L-Carnitine-Tartrate, caffeine and a combination thereof. Each possibility represents a separate embodiment of the present invention.

It is to be noted, that according to some embodiments of the present invention mitochondria and/or at least one mitochondrial constituent are administered together with at least one hair-growth inducing agent. However, the present invention precludes administration of known hair-growth inducing agents by themselves, such as, but not limited to: Finasteride, Dutasteride, Minoxidil, Kopexil, oxidized coenzyme Q, reduced coenzyme Q, L-Carnitine-Tartrate, caffeine and a combination thereof.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Example 1: Micro-Dissected Human Hair Follicles (hHFs) Show Hair Follicle Elongation in the Presence of Freeze-Thawed Human Mitochondria Compositions

Mitochondria were isolated from human term placenta according to the following protocol:

1. Placenta was rinsed free of blood by using ice-cold TB buffer (isolation buffer: 200 mM sucrose, 1 mM EGTA and 10 mM Tris-MOPS)+0.2% BSA.
2. The placenta was minced into small pieces in 5 ml IB+0.2% BSA using scissors.
3. The suspension was transferred to a 10 ml glass potter and homogenized using a Dounce glass homogenizer by five complete up and down cycles.
4. The homogenate was transferred to a 15 ml tube and centrifuged at 600 g for 10 min at 4° C.
5. The supernatant was transferred to clean centrifuge tubes and the pellet was resuspended in IB buffer, and subjected to a second centrifugation step.
6. The supernatant from steps 4 and 5 was filtered through a 5 μm filter to remove any cells or large cell debris.
7. The supernatant was recovered and centrifuged at 7,000×g for 15 min.
8. The mitochondrial pellet was washed in 10 ml ice cold IB buffer and mitochondria were recovered by centrifugation at 7,000×g for 15 min at 4° C.
9. The supernatant was discarded and the pellet resuspended, containing mitochondria in 200 μl of IB buffer.
10. Protein content was determined by the Bradford assay.

The mitochondria were flash-frozen in IB (200 mM sucrose, 1 mM EGTA and 10 mM Tris-MOPS) in 1.5 ml Eppendorf tubes at a concentration of 2 μgr/μl. The mitochondria were kept at −80° C. for 2 months and thawed quickly by hand prior to use.

Scalp skin samples containing mainly human hair follicles (hHFs) in anagen subphase VI (in which a new hair shaft emerges from the skin surface) have been obtained from a male who underwent an elective face lift plastic surgery (informed consent has been obtained). The hair follicles were micro-dissected and initially cultured for 1 day (day 0-1) in hHFs culture medium without stimuli in order to verify their growth potential, thus allowing selection of the most appropriate hair follicles for the experiment.

Next, the hHFs were cultured for 1 day (day 1-2) in hHFs culture medium supplemented with 5, 12.5 or 50 μg/ml of the thawed mitochondria or Cyclosporine A (a known inducer of hHFs growth) as a positive control. Twelve hair follicles have been used for each one of the treatments. All treatments were given once, except for the hHFs treated with 5 μg/ml mitochondria composition, which received a second treatment for one day (day 5-6) after 5 days. The hHFs culture medium was replaced every other day for all treatments.

At day 5 and day 9 the wells containing the hHFs were photographed and the elongation of each hair follicle was measured (in millimeters). Image analysis has been performed by the ImageJ software (NIH, USA).

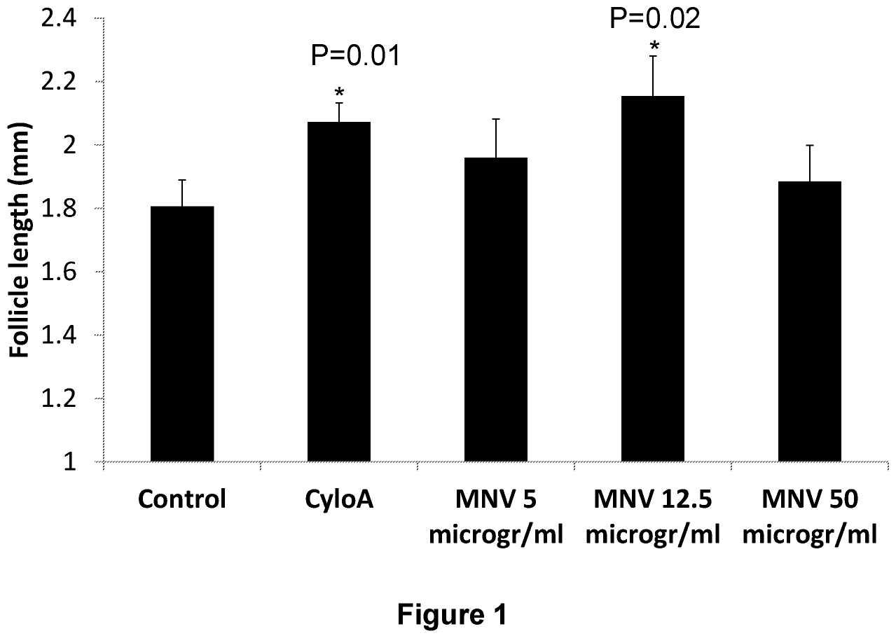

As depicted in FIG. 1, the growth percentage of hHFs in each treatment group was measured relatively to the growth of untreated hHFs incubated in hHFs medium (marked “Vehicle” in FIG. 1). All hHFs treated with the thawed mitochondria showed higher growth compared to hHFs incubated in hHFs medium. The hHFs treated with 12.5 μg/ml of the thawed mitochondria showed the highest elongation (19%).

### Example 2: Incubation of Human Follicular Dermal Papilla Cells with Fresh Mitochondria Results in Higher Cell Proliferation

Human follicular dermal papilla cells (PromoCell) were seeded in 24 wells plates (60,000 cells/well). The cells were treated with 5 μg human placental mitochondria at a concentration of 12.5 μg/ml. The mitochondria were produced as in Example 1, without the freeze-thaw cycle. Following an incubation of 24 hours the medium was replaced and cells were grown for additional 5 days.

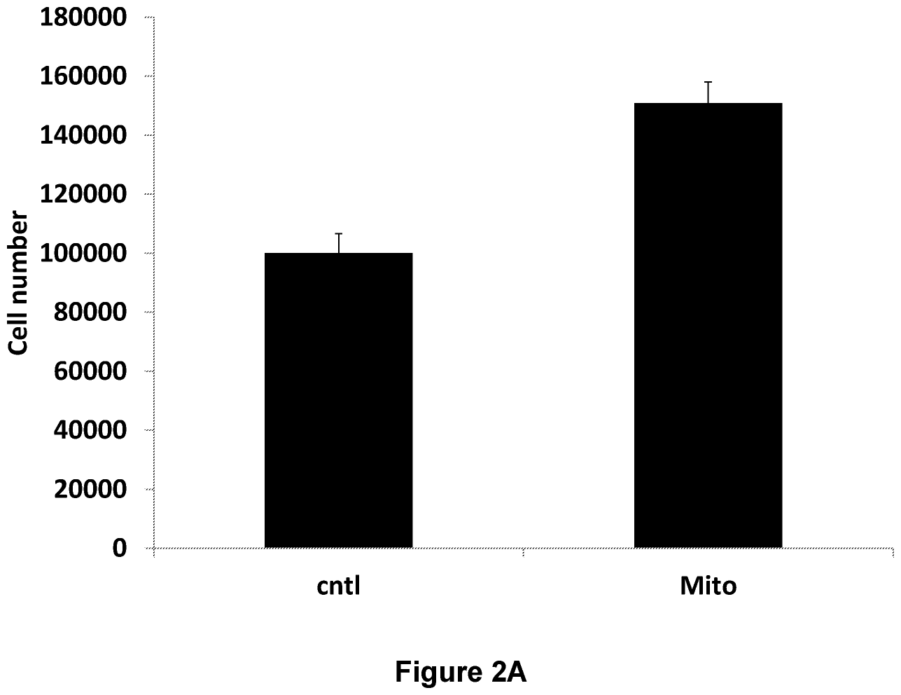

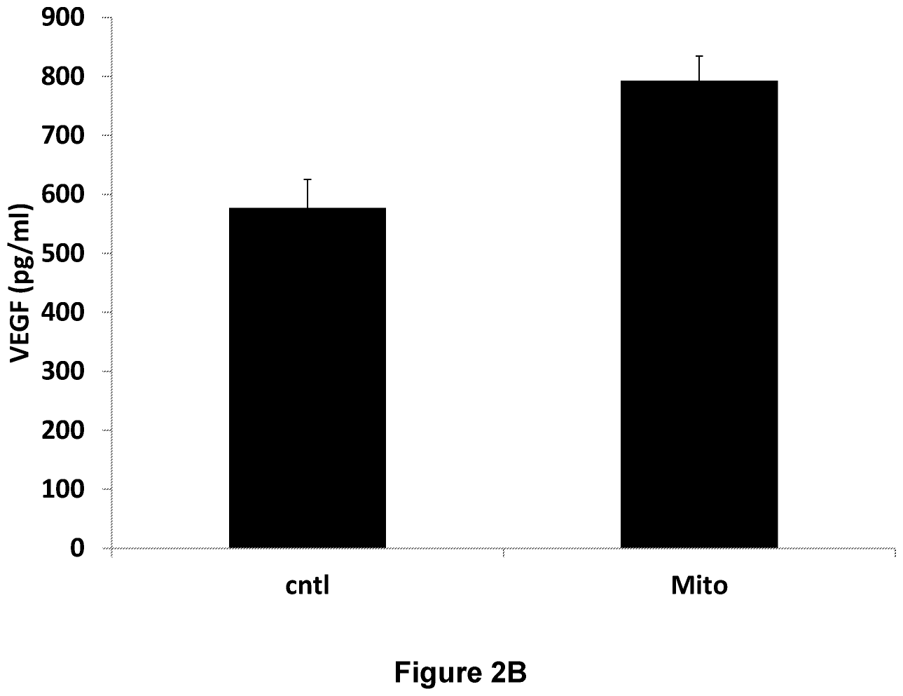

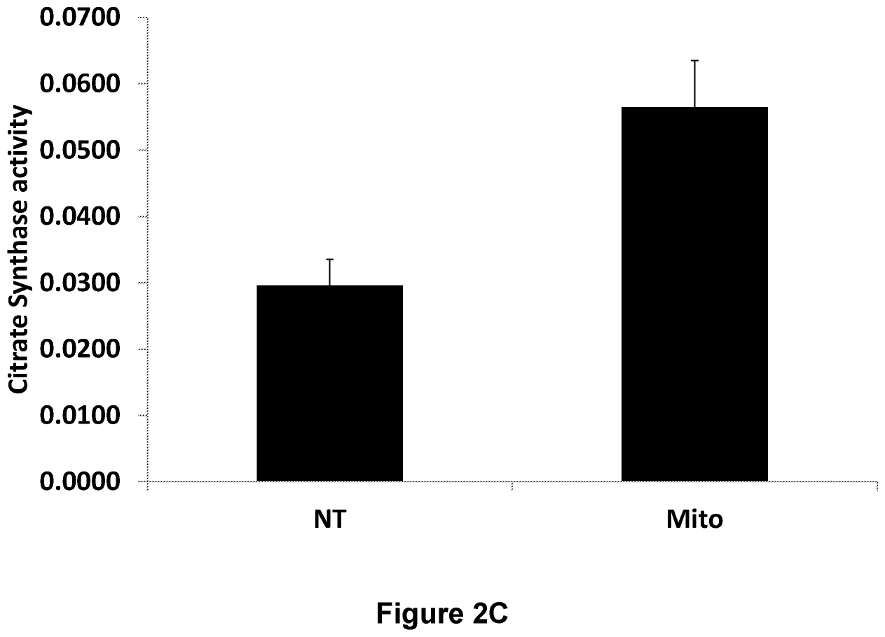

As can be seen in FIG. 2A, cell number was higher in cells that were treated with the mitochondria. As known in the art, human follicular dermal papilla cells produce VEGF-A (Lachgar S. et al., Vascular Endothelial Growth Factor is an autocrine growth factor for hair dermal papilla cells, Journal of Investigative Dermatology, 1996, (106):17-23). As can be seen in FIG. 2B, cells that were treated with mitochondria secrete more VEGF-A to the medium, in correlation with the higher cell number (VEGF-A levels were evaluated using an ELISA kit by R&R Systems). As can be seen in FIG. 2C, the level of citrate synthase activity was higher in cells treated with mitochondria for 3 hours.

### Example 3: Mitochondria that were Frozen and Thawed Show Oxygen Consumption Comparable to that of Non-Frozen Mitochondria

Mitochondria were isolated from mouse term placenta according to the following protocol:

1. Placenta was rinsed free of blood by using ice-cold IB buffer (isolation buffer: 200 mM sucrose, 1 mM EGTA and 10 mM Tris-MOPS)+0.2% BSA.
2. The placenta was minced into small pieces in 5 ml IB+0.2% BSA using scissors.
3. The suspension was transferred to a 10 ml glass potter and homogenized using a Dounce glass homogenizer by five complete up and down cycles.
4. The homogenate was transferred to a 15 ml tube and centrifuged at 600 g for 10 min at 4° C.
5. The supernatant was transferred to clean centrifuge tubes and the pellet was resuspended in IB buffer, and subjected to a second centrifugation step.
6. The supernatant from steps 4 and 5 was filtered through a 5 μm filter to remove any cells or large cell debris.
7. The supernatant was recovered and centrifuged at 7,000×g for 15 min.
8. The mitochondrial pellet was washed in 10 ml ice cold IB buffer and mitochondria were recovered by centrifugation at 7,000×g for 15 min at 4° C.
9. The supernatant was discarded and the pellet resuspended, containing mitochondria in 200 μl of IB buffer.
10. Protein content was determined by the Bradford assay.

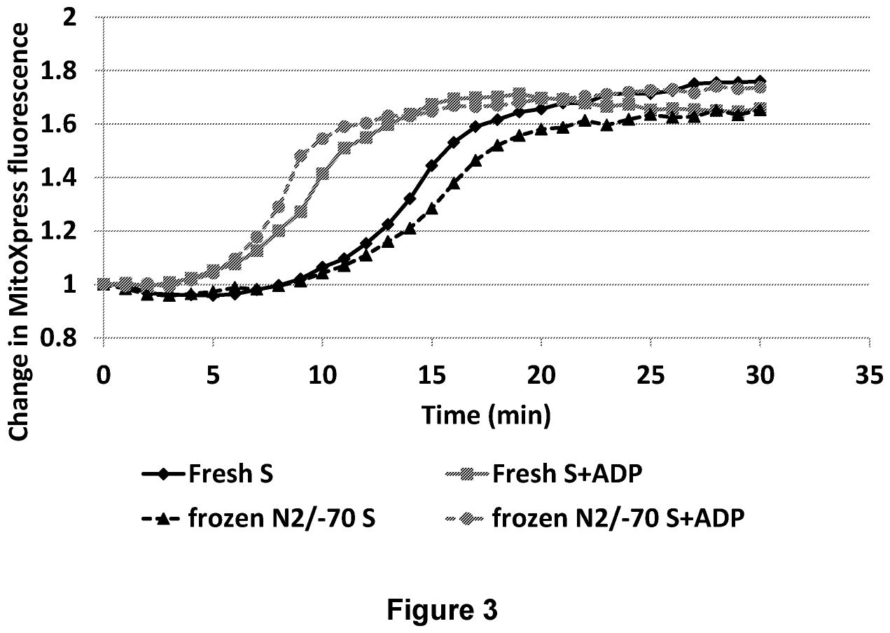

To compare activity of frozen versus unfrozen mitochondria, mitochondria were flash-frozen using liquid nitrogen in IB (200 mM sucrose, 1 mM EGTA and 10 mM Tris-MOPS) in 1.5 ml Eppendorf tubes and kept at −70° C. for 30 minutes. Mitochondria were thawed quickly by hand and O₂ consumption by 100 μg mitochondria was measured using the MitoXpress fluorescence probe (Luxcel) and a Tecan plate reader. Oxygen consumption was measured in the presence of 25 mM Succinate (S) or in the presence of 25 mM Succinate and 1.65 mM ADP (S+ADP). The change in fluorescence was calculated relative to the level of fluorescence at time 0. FIG. 3 shows that the O₂ consumption, and rate of O₂ consumption, were comparable for mitochondria that were frozen and thawed (marked “Frozen”) in comparison to non-frozen mitochondria (marked “Fresh”).

As opposed to frozen mitochondria, mouse placental mitochondria that were chilled (kept for 4 days at 4° C.) produced less ATP than fresh mitochondria (Table 1).

TABLE 1
ATP production of fresh and chilled mouse placental mitochondria

| | ATP (RLU) |
|---|---|
| Fresh Mitochondria (F) | 4690 |
| Chilled Mitochondria (C) | 1587 |

### Example 4: Comparison of Oxygen Consumption in Fresh Vs. Thawed Potato-Derived Mitochondria Incubated in a Sucrose or Mannitol Containing Buffer

Mitochondria were isolated from 30 grams of potato in a buffer comprising mannitol (0.7 M mannitol, 10 mM KPI (pH 6.5), 1 μM EDTA, 2 μM Cystein, supplemented with 0.1% fatty acid free BSA) or an isolation buffer (IB) comprising sucrose (200 mM Sucrose, 1 mM EGTA/Tris pH 7.4, 10 mM Tris/Mops pH 7.4, supplemented with 0.2% fatty acid free BSA). Briefly, the potatoes were chilled over-night at 4° C., cut into small pieces and pulverized using a blender in the mannitol or sucrose containing buffer (at a 1:4 ratio of tissue:volume) for 30 seconds. The mixtures were filtered through a cheese cloth and centrifuged at 600 g for 10 minutes at 4° C. The supernatants were transferred to new tubes and centrifuged at 8000 g for 10 minutes. The pellets of the mannitol/sucrose treated tissues were washed with 1 ml wash buffer (0.7 M Mannitol, 10 mM KPI pH 6.5) or isolation buffer, respectively, centrifuged at 8000 g for 10 minutes at 4° C. and re-suspended in 100 μl wash buffer/isolation buffer.

Oxygen consumption was measured in 50 μg of fresh mitochondria or mitochondria that had been snap-frozen in liquid nitrogen, kept 24 hours in liquid nitrogen and thawed at room temperature using the MitoXpress fluorescence probe (Luxcel) and a Tecan plate reader, in the presence of succinate (S), succinate+ADP (S+A), glutamate and malate (G/M) or glutamate, malate and ADP (G/M+ADP).

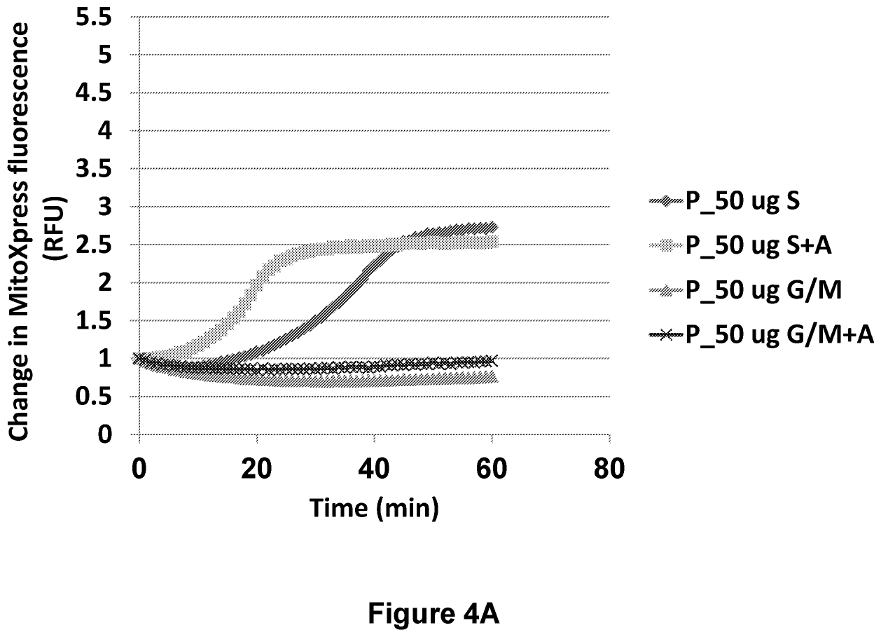

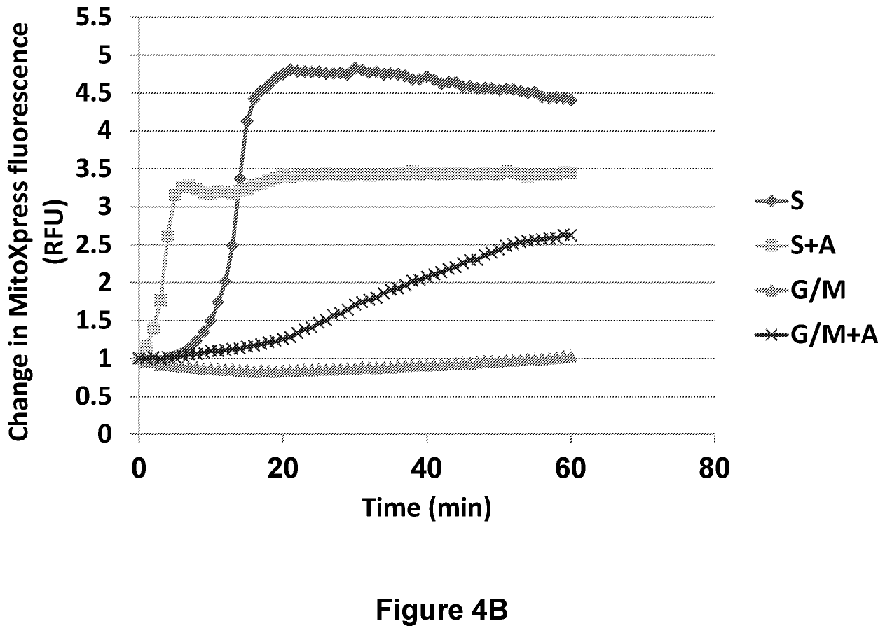

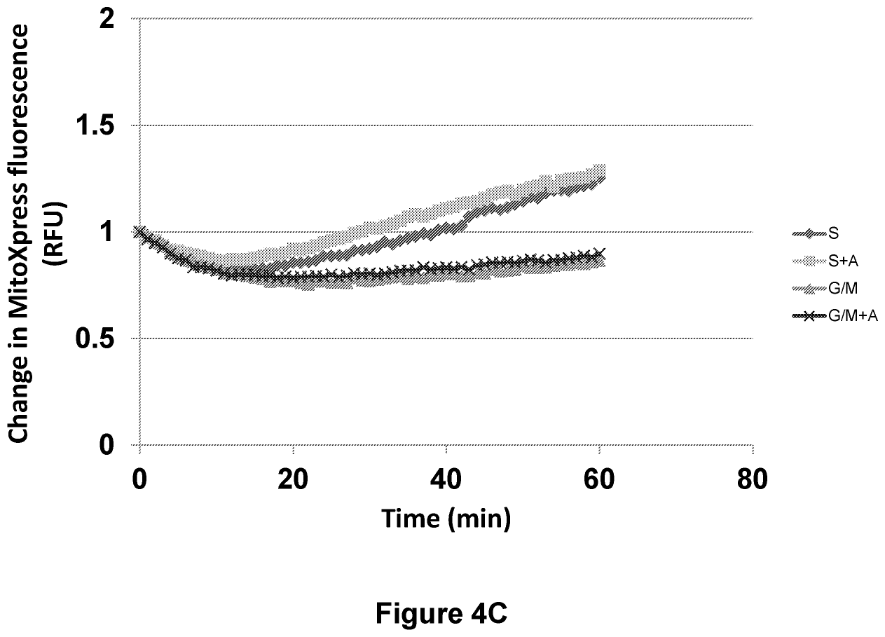

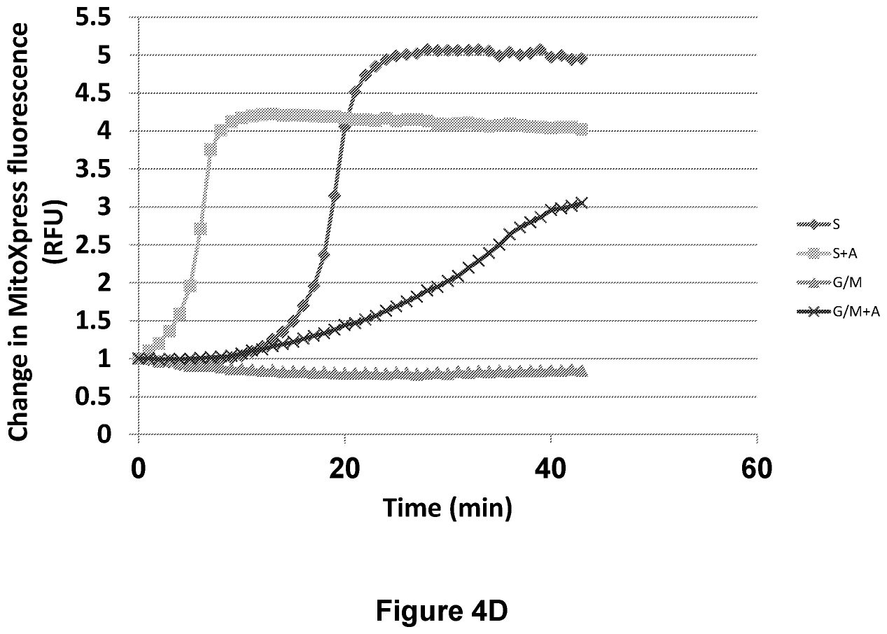

As can be seen in FIG. 4, fresh mitochondria incubated in a mannitol comprising buffer (FIG. 4A) showed lower and slower oxygen consumption than fresh mitochondria incubated in a buffer comprising sucrose (FIG. 4B). Mitochondria frozen and thawed in a buffer comprising mannitol (FIG. 4C) did not show oxygen consumption. Mitochondria frozen and thawed in a buffer comprising sucrose (FIG. 4D) showed comparable oxygen consumption to the corresponding fresh mitochondria (FIG. 4B).

### Example 5: Comparison of Membrane Integrity in Fresh Vs. Thawed Potato-Derived Mitochondria Incubated in a Sucrose or Mannitol Containing Buffer

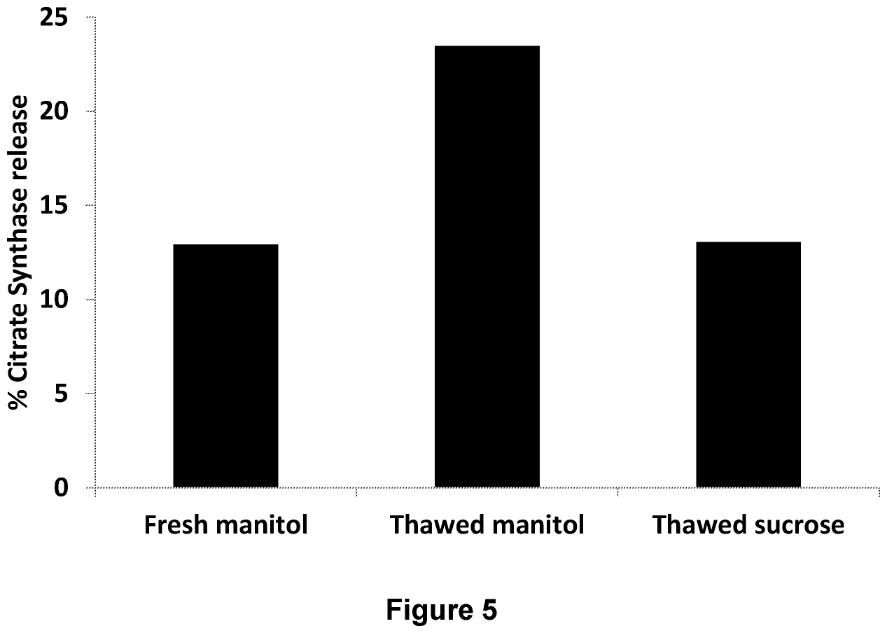

Mitochondria were isolated from potatoes and treated as described in Example 4. Next, 50 μg of mitochondria homogenate were centrifuged at 7000 g for 10 min, the supernatant was collected into a new tube and the pellet was re-suspended in lysis buffer. In order to assess the integrity of the mitochondrial inner membrane, 16 μg of mitochondria supernatant and 4 μg of mitochondria pellet were examined for release of citrate synthase using kit CS0720 (Sigma). FIG. 5 demonstrates citrate synthase release of mitochondria incubated with sucrose or mannitol, either fresh or following a freeze/thaw cycle. FIG. 5 shows that mitochondria that were isolated and frozen in mannitol containing buffer have decreased membrane integrity, as witnessed by citrate synthase release.

### Example 6: Comparison of Oxygen Consumption and Membrane Integrity of Mitochondria Incubated in Isolation Buffer Vs. Mitochondria Incubated in PBS

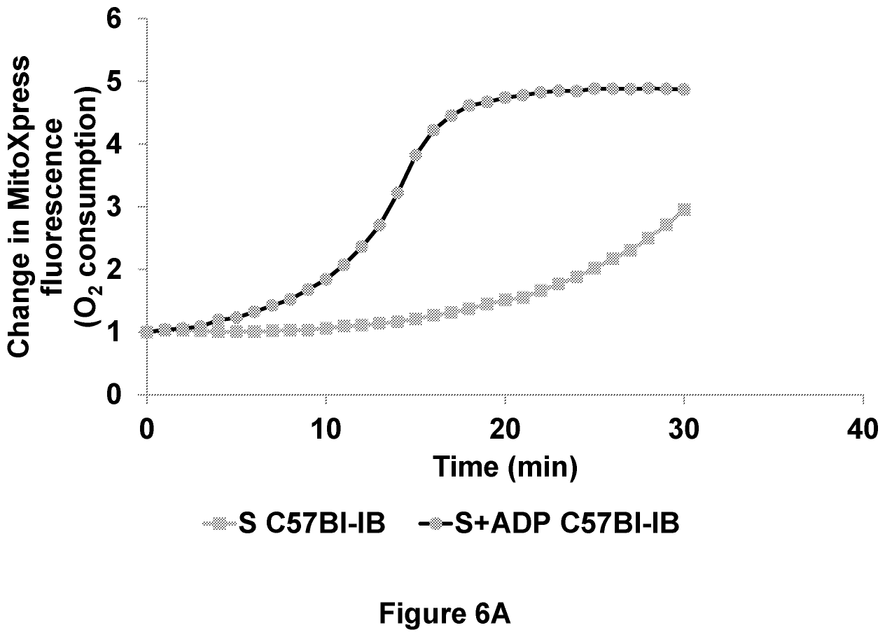

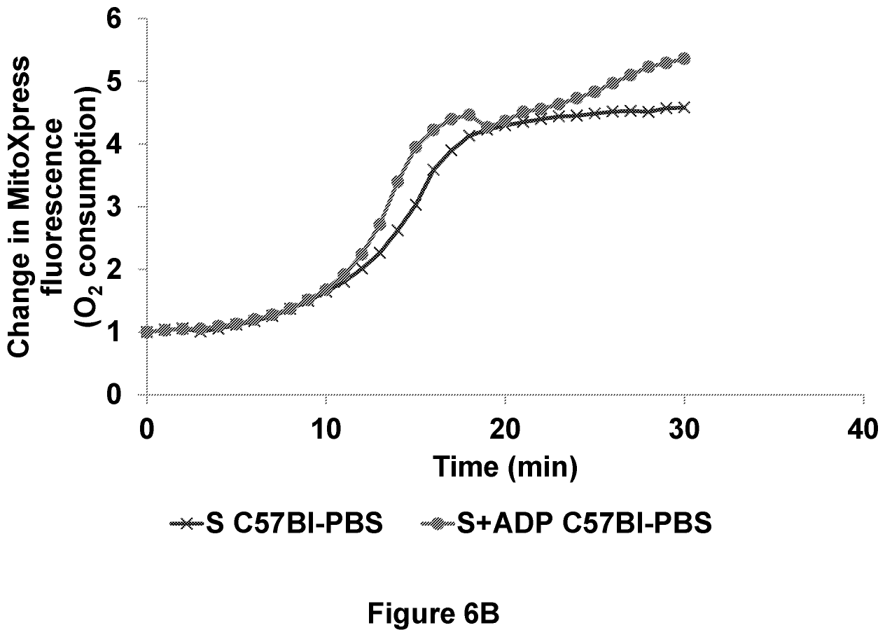

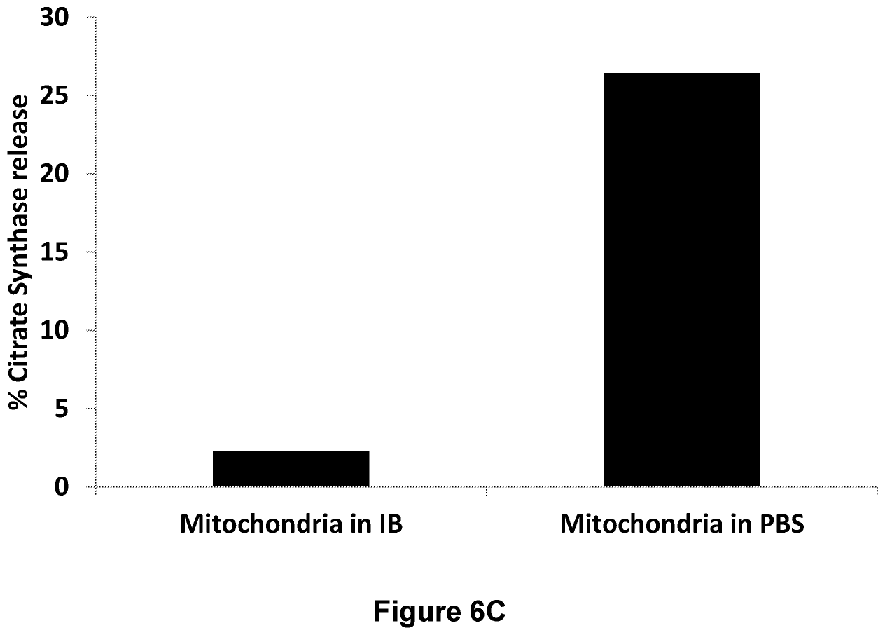

Mitochondria were isolated from mouse term placenta using isolation buffer (IB) (200 mM Sucrose, 1 mM EGTA/Tris pH 7.4, 10 mM Tris/Mops pH 7.4 supplemented with 0.2% fatty acid free BSA). The mitochondria pellet was either suspended in IB and incubated on ice, or suspended in PBS and incubated at 37° C. for 10 min. Oxygen consumption was measured for 50 μg mitochondria incubated in the presence of succinate (S) or succinate+ADP (S+A) using the MitoXpress fluorescence probe (Luxcel). As can be seen in FIG. 6, mitochondria that have been incubated with PBS (FIG. 6B) show oxygen consumption corresponding to un-coupled mitochondria, while mitochondria incubated in IB (FIG. 6A) show oxygen consumption corresponding to coupled mitochondria.

Mitochondrial inner membrane integrity of mitochondria incubated in IB was compared to that of mitochondria incubated in PBS by measuring citrate synthase release using the CS0720 kit (Sigma). FIG. 6C shows that mitochondria that were incubated in PBS have decreased membrane integrity, as witnessed by citrate synthase release.

### Example 7: Comparison of Oxygen Consumption and Membrane Integrity of Mitochondria Incubated in Isolation Buffer Vs. Mitochondria Incubated in Cell Culture Medium

Mitochondria were isolated from mouse term placenta using isolation buffer (IB) (200 mM Sucrose, 1 mM EGTA/Tris pH 7.4, 10 mM Tris/Mops pH 7.4 supplemented with 0.2% fatty acid free BSA). The mitochondria pellet was suspended for 1 hour either in IB on ice or in OptiMEM cell medium (Gibco; 2.5 gr/L glucose=−13.8 mM) at 37° C.

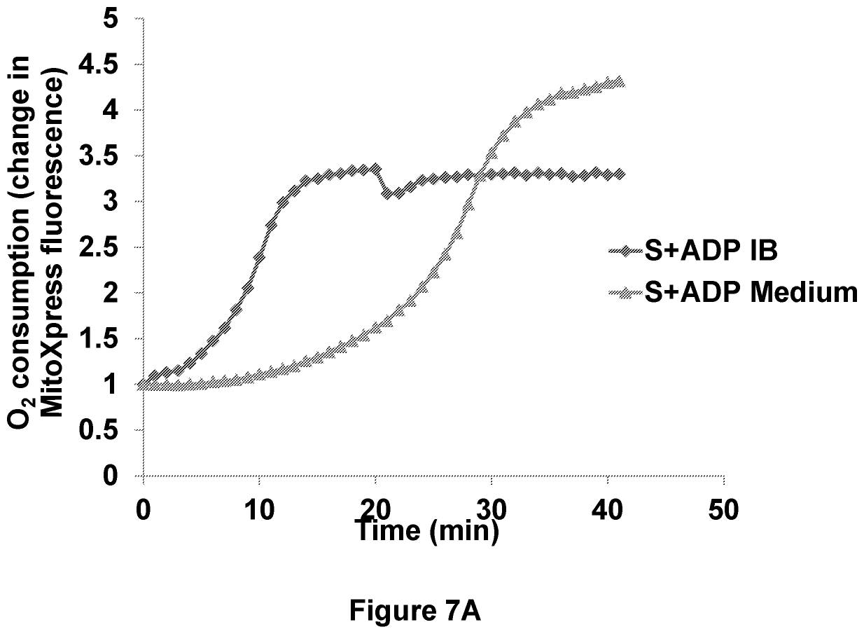

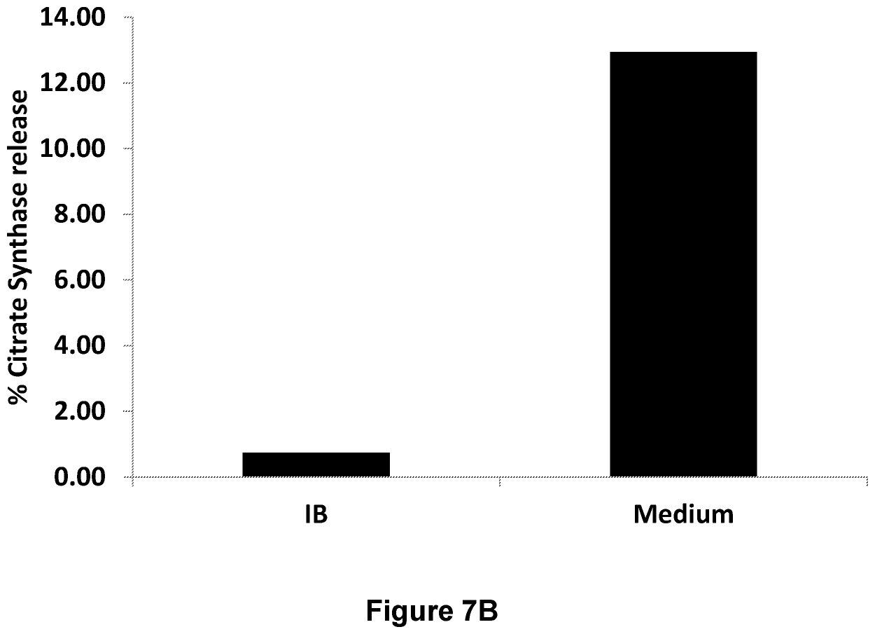

Oxygen consumption was measured for 50 μg mitochondria incubated in the presence of succinate+ADP (S+A) using the MitoXpress fluorescence probe (Luxcel). FIG. 7A shows that mitochondria that have been incubated in medium show oxygen consumption corresponding to un-coupled mitochondria, while mitochondria incubated in IB show oxygen consumption corresponding to coupled mitochondria.

Mitochondrial inner membrane integrity of mitochondria incubated in IB was compared to that of mitochondria incubated in medium by measuring citrate synthase release using the CS0720 kit (Sigma). FIG. 7B shows that mitochondria that were incubated in medium have decreased membrane integrity, as witnessed by citrate synthase release.

### Example 8: Placental Mitochondria are Able to Produce Progesterone

Mitochondria were isolated from human term placenta according to the following protocol:

1. Placenta was rinsed free of blood by using ice-cold M1 buffer (isolation buffer: 200 mM sucrose, 1 mM EGTA and 10 mM Tris-MOPS)+0.2% BSA.
2. The placenta was minced into small pieces in 5 ml M1+0.2% BSA using scissors.
3. The suspension was transferred to a 10 ml glass potter and homogenized using a Dounce glass homogenizer by five complete up and down cycles.
4. The homogenate was transferred to a 15 ml tube and centrifuged at 600 g for 10 min at 4° C.
5. The supernatant was transferred to clean centrifuge tubes and the pellet was resuspended in M1 buffer, and subjected to a second centrifugation step.
6. The supernatant from steps 4 and 5 was filtered through a 5 μm filter to remove any cells or large cell debris.
7. The supernatant was recovered and centrifuged at 7,000×g for 15 min.
8. The mitochondrial pellet was washed in 10 ml ice cold M1 buffer and mitochondria were recovered by centrifugation at 7,000×g for 15 min at 4° C.
9. The supernatant was discarded and the pellet resuspended, containing mitochondria in 200 μl of M1 buffer.
10. Protein content was determined by the Bradford assay.

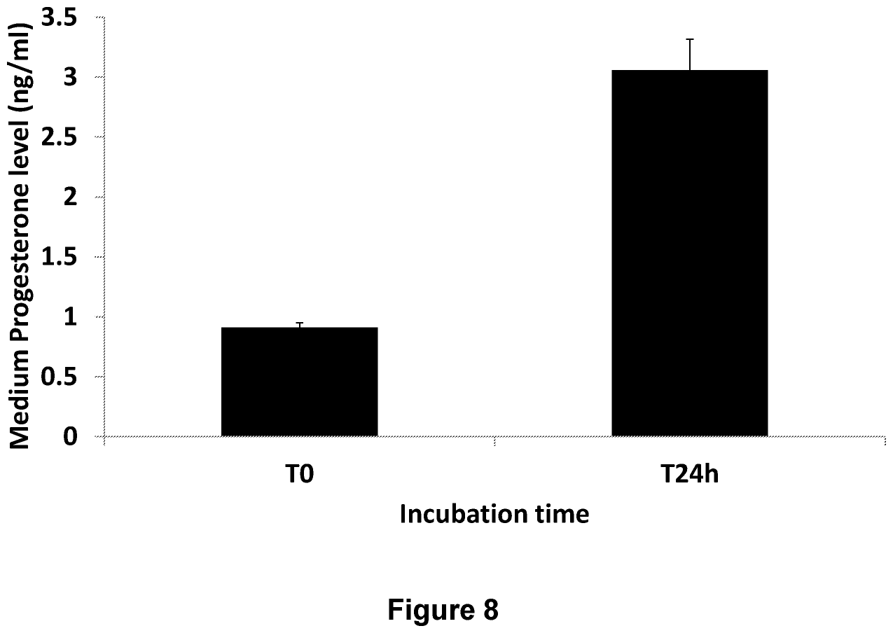

To verify functionality of isolated mitochondria, their ability to produce progesterone was examined (a unique feature of functional term placental mitochondria). Mitochondria (50 μg) were incubated in 200 μl of fetal calf serum at 37° C., 5% CO₂ for 24 h (T24 h). The level of progesterone secretion to the media was determined by radio immune assay (RIA) in A. M. L. (Herzelia, Israel). As can be seen in FIG. 8, the level of progesterone in the media was higher after 24 h than at T0, indicating that mitochondria that were incubated for 24 h show higher progesterone production.

### Example 9: Human Follicle Dermal Papilla Cells (hFDPCs) Show Increase in ATP Content in the Presence of Fresh Sprout Mitochondria Compositions

Mitochondria were isolated from sprouts according to the following protocol:

1. 400 gram of Vigna Radiata sprouts were washed and minced.

### 2. Homogenization in 2 L of Sucrose Buffer (250 mM Sucrose, 10 Mm Tris/HCl, 1 mM EDTA, pH 7.4).

### 3. Centrifugation at 600 g, 4° C.

4. Filter by 5 μm cutoff.

### 5. Centrifugation at 8000 g, 4° C.

6. Pellet wash and Centrifugation at 8000 g, 4° C.

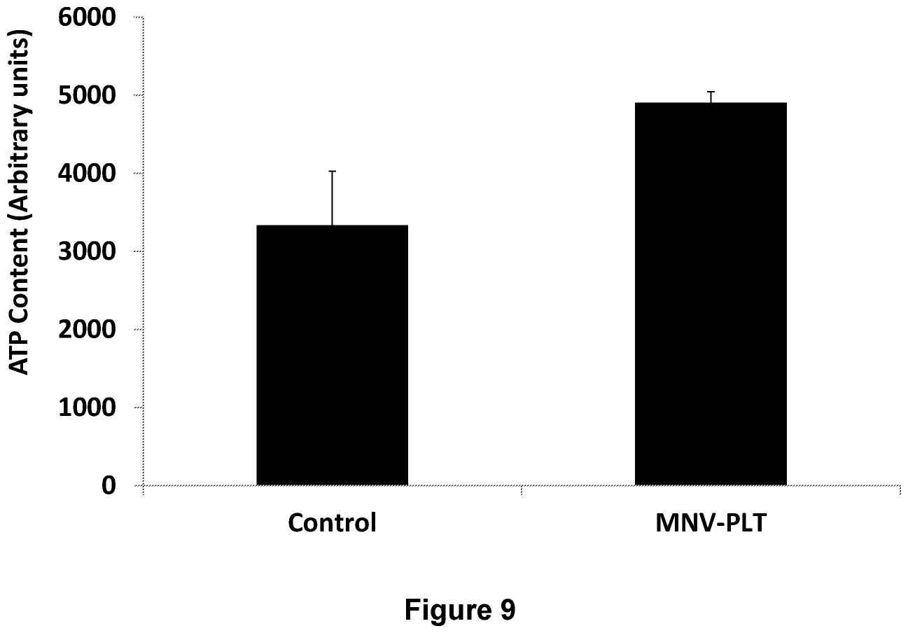

As can be seen in FIG. 9, the level of ATP in naïve hFDPCs (Control) is significantly (p<0.05) increased by sprout mitochondria (MNV-PLT).

### Example 10: Human Follicle Dermal Papilla Cells (hFDPCs) Show Increase in Citrate Synthase (CS) Enzyme Activity, Proliferation and VEGF Secretion in the Presence of Human Placenta Mitochondria Compositions

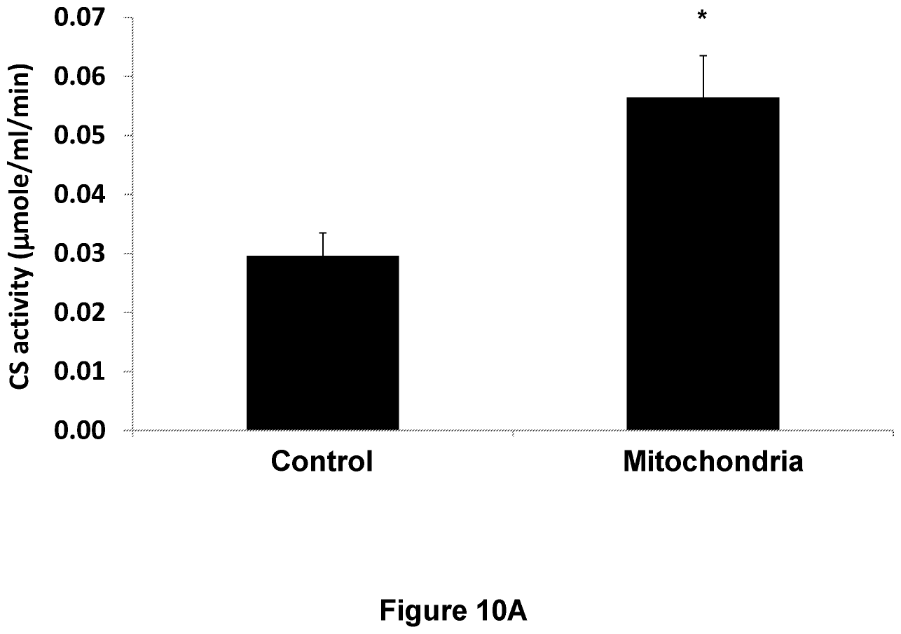

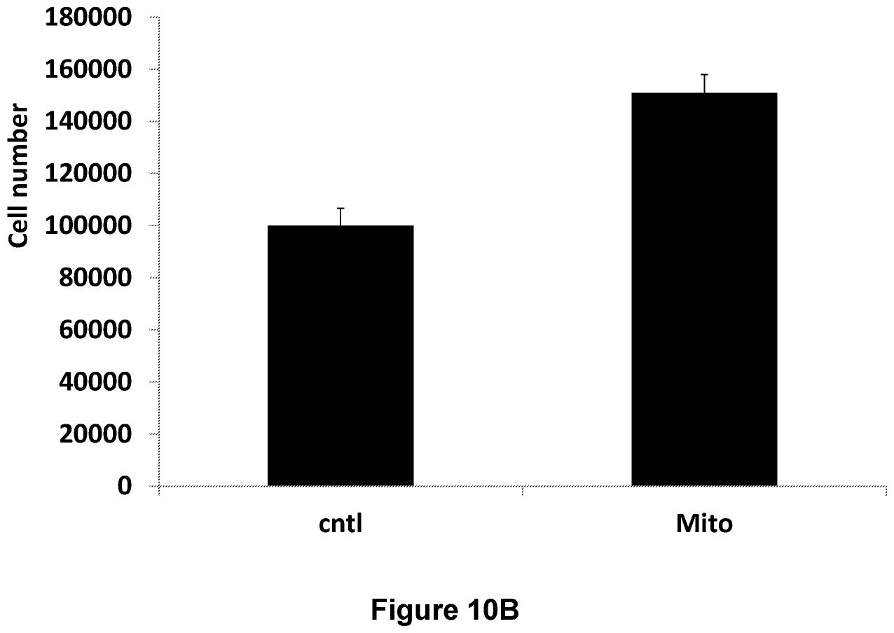

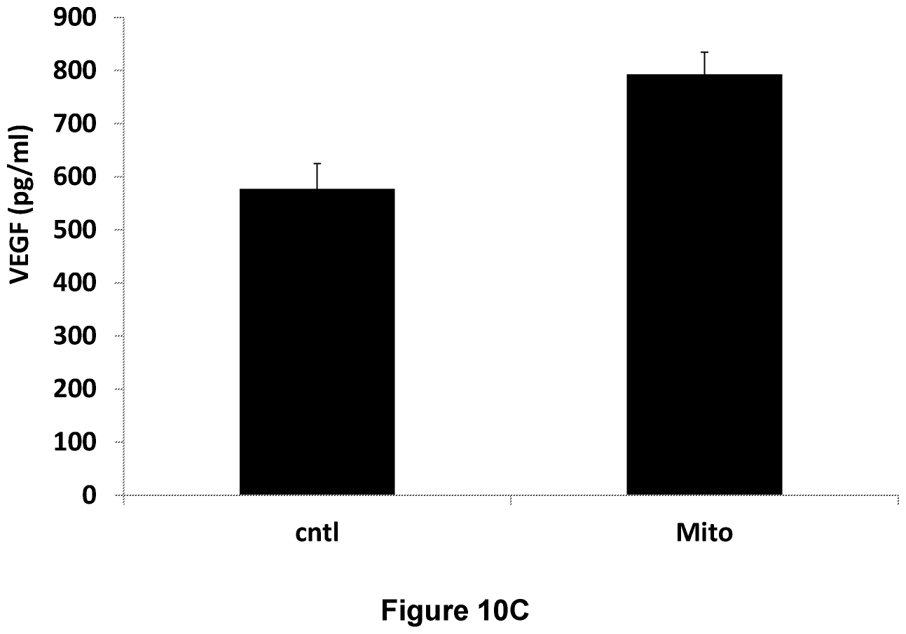

The mitochondria were produced as in Example 8, but using Sucrose Buffer (250 mM Sucrose, 10 Mm Tris/HCl, 1 mM EDTA, pH 7.4). As can be seen in FIGS. 10A-10C, citrate synthase (CS) enzyme activity, hFDPCs proliferation, and hFDPCs VEGF secretion are all significantly (p<0.05) increased in the presence of human placenta mitochondria compositions.

### Example 11: Human Skin is Protected from UV-B and ROS in the Presence of Fresh Sprout Mitochondria Compositions

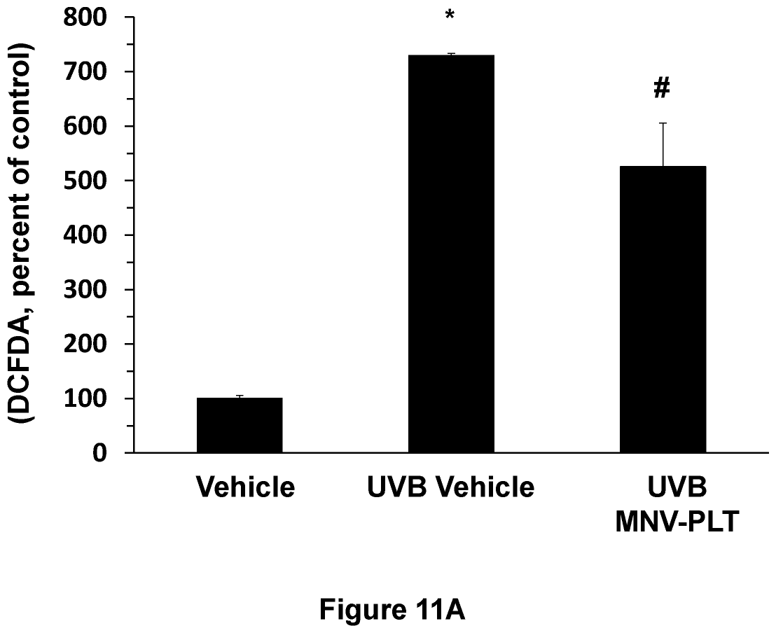

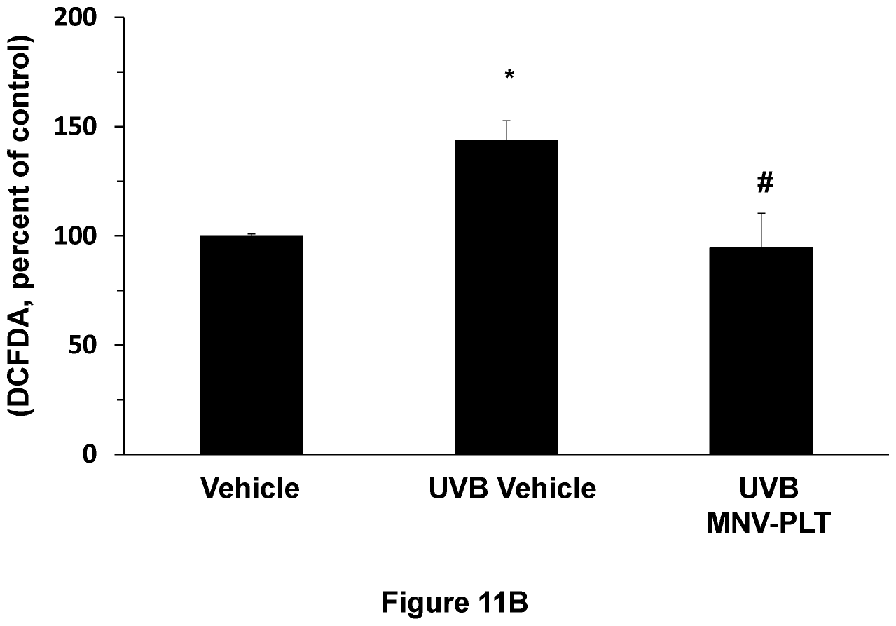

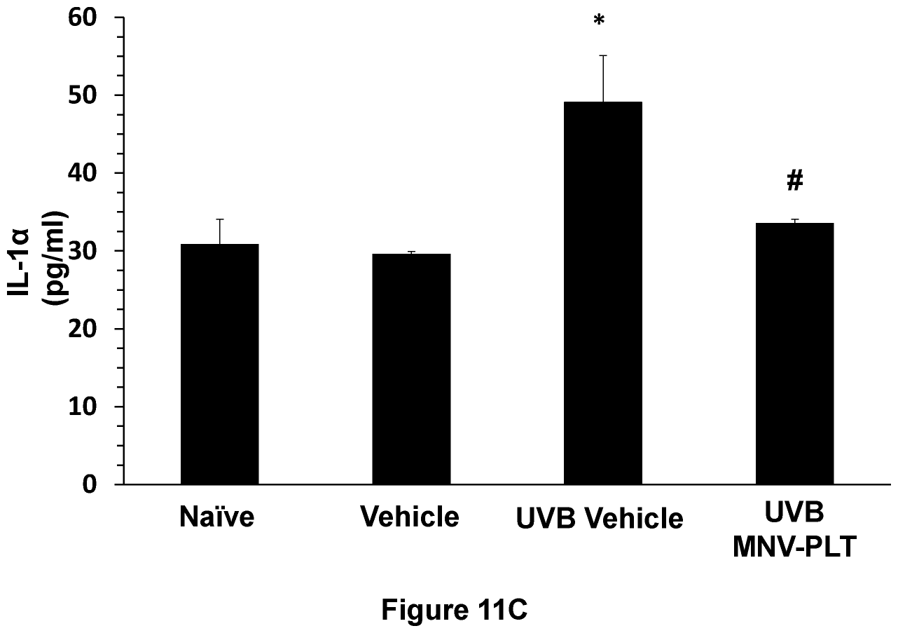

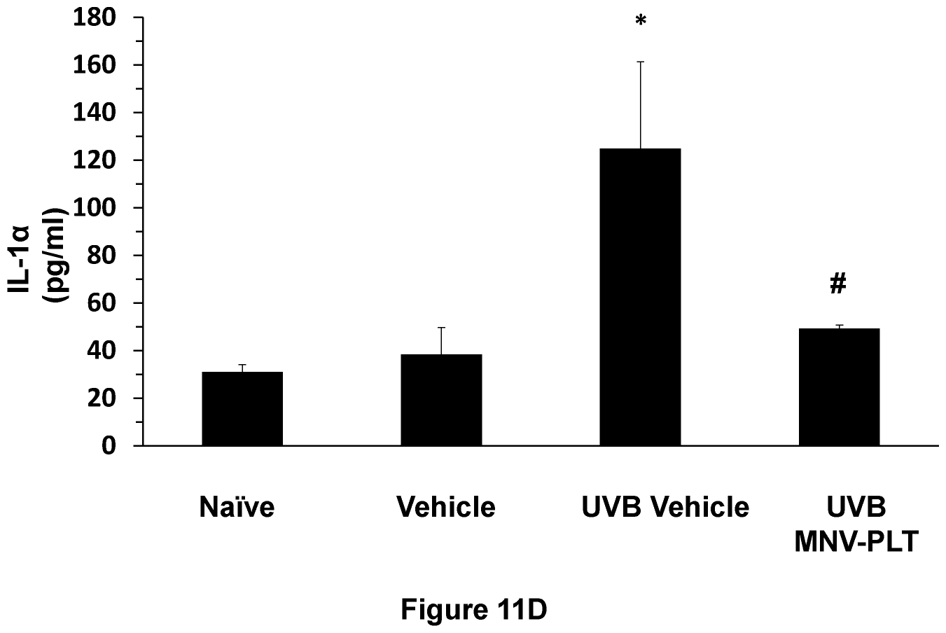

The mitochondria were produced as in Example 9. As can be seen in FIGS. 11A-11D, UV-B radiation increased the production of ROS (FIG. 11A and FIG. 11B) and IL-la (FIG. 11C and FIG. 11D) in skin cells, while sprout mitochondria were able to significantly (p<0.05) reduce these effects, both when added to the medium of the cells (FIG. 11A and FIG. 11C) and when topically-applied to the cells (FIG. 11B and FIG. 11D).

### Example 12: Improved Hair Vitality in a 7-Year-Old Boy Afflicted with Pearson Syndrome

A 7-years old male patient was diagnosed with Pearson Syndrome, having a deletion of nucleotides 5835-9753 in his mtDNA. The patient received a single round of treatment of autologous CD34+ cells enriched ex-vivo with healthy mitochondria from his mother. The CD34+ cells were prepared by incubating naïve CD34+ cells with healthy mitochondria, which resulted in a 1.6 fold increase in the cells' mitochondrial content (60% increase in mitochondrial content as demonstrated by CS activity). Unexpectedly, despite having gone through only a single round of treatment, the patient stopped from shedding his hair and surprisingly managed to regrow full hair on his head.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-39. (canceled)

40. A method for preventing, ameliorating or treating hair loss, the method comprising administering to a subject in need thereof a composition comprising an effective amount of about 1 μg/ml to about 100 μg/ml of intact mitochondria, ruptured mitochondria and/or mitochondrial constituents selected from the group consisting of mitochondrial protein, mitochondrial nucleic acid, mitochondrial lipid and mitochondrial saccharide.

41. The method of claim 40, wherein the treatment is selected from the group consisting of stopping hair follicle miniaturization, slowing hair follicle miniaturization, reversing hair follicle miniaturization, inducing elongation of hair follicles, enhancing elongation of hair follicles, inducing proliferation of cells within hair follicles, enhancing proliferation of cells within hair follicles, inducing elongation of hair fibers, enhancing elongation of hair fibers, enhancing thickness of hair fibers, altering the duration of hair follicle growth cycle phases and any combination thereof.

42. The method of claim 40, wherein the composition comprises about 5 μg/ml to about 50 μg/ml of the mitochondrial constituents.

43. The method of claim 40, wherein the intact mitochondria, ruptured mitochondria and/or mitochondrial constituents are frozen-thawed.

44. The method of claim 40, wherein the composition is formulated as a colloid, a liquid, a lotion, a cream, an ointment, foam or gel.

45. The method of claim 40, wherein said mitochondria are derived from a cell or a tissue selected from the group consisting of placenta, placental cells grown in culture, blood cells, plant tissue, plant cells and plant cells grown in culture.

46. The method of claim 40, wherein the composition is administered by topical administration, oral administration, subcutaneous administration, intradermal administration, transdermal administration or systemic administration.

47. The method of claim 46, wherein the composition is administered to a human scalp.

48. The method of claim 40, wherein the subject is afflicted with a disease, disorder or condition which has a deleterious effect on hair vitality.

49. The method of claim 48, wherein the disease, disorder or condition is selected from the group consisting of alopecia, mitochondrial disease and an autoimmune disorder.

50. The method of claim 48, wherein the subject is afflicted with cancer and is treated by radiation or chemotherapy.

51. The method of claim 40, wherein the subject is over 30, over 40, over 50 or over 60 years of age.

52. The method of claim 40, wherein the subject is a male.

53. A cosmetic composition for preventing, ameliorating or treating hair loss, wherein the composition comprises an effective amount of about 1 μg/ml to about 100 μg/ml of intact mitochondria, ruptured mitochondria and/or mitochondrial constituents selected from the group consisting of mitochondrial protein, mitochondrial nucleic acid, mitochondrial lipid and mitochondrial saccharide, wherein one or more of the intact mitochondria, the ruptured mitochondria and/or the mitochondrial constituents is frozen or thawed, and wherein the composition further comprises a cosmetically-acceptable carrier and is formulated for topical administration to human skin.

54. The cosmetic composition of claim 53, wherein the composition comprises sucrose.

55. The cosmetic composition of claim 53, wherein the intact mitochondria, ruptured mitochondria and/or mitochondrial constituents are obtainable or obtained by a method comprising the steps (a)-(d):
(a) obtaining a sample of a human or plant tissue or organ,
(b) homogenizing the tissue or organ,
(c) isolating the liquid phase, and
(d) isolating intact mitochondria, ruptured mitochondria and/or mitochondrial constituents from the liquid phase.

56. The cosmetic composition of claim 53, wherein the intact mitochondria, ruptured mitochondria and/or mitochondrial constituents are obtainable or obtained from a cell or a tissue selected from the group consisting of placenta, placental cells grown in culture, blood cells, plant tissue, plant cells and plant cells grown in culture.

57. The cosmetic composition of claim 56, wherein the placenta, placental cells grown in culture or blood cells are human.

58. The cosmetic composition of claim 56, wherein the plant cells are sprout cells or potato cells.

59. A lotion, cream, ointment, foam, gel, soap, shampoo or conditioner, comprising the cosmetic composition of claim 53.
